(19) United States
(12) Patent Application Publication
(10) Pub. No.: US 2007/0254899 A1
(43) Pub. Date: Nov. 1, 2007

(54) Soluble salts of thieno[2,3-d]pyrimidine derivatives

(75) Inventors: Martin Ian Cooper, Cambridgeshire (GB); Christopher Stephen Frampton, Suffolk (GB)

Correspondence Address: LAHIVE & COCKFIELD, LLP, ONE POST OFFICE SQUARE, BOSTON, MA, 02109-2127, US

(73) Assignee: Dynogen Pharmaceuticals, Inc., Waltham, MA (US)

(21) Appl. No.: 11/728,966

(22) Filed: Mar. 27, 2007

Related U.S. Application Data
(60) Provisional application No. 60/788,565, filed on Mar. 31, 2006.
(60) Provisional application No. 60/808,905, filed on May 26, 2006.

(51) Int. Cl. A61K 31/519; A61P 1/00; A61P 1/08; A61P 1/12; A61P 13/10; C07D 495/02

(52) U.S. Cl. 514/260.1; 544/278

(57) ABSTRACT

The present invention is directed to novel salts of thieno[2,3-d]pyrimidine derivatives, including 4-(2-fluorophenyl)-6-methyl-2-(piperazin-1-yl)thieno[2,3-d]pyrimidine salts. The present invention is also directed to compositions including such polymorphs and methods for using such salts, e.g., in the treatment of gastrointestinal and/or genitourinary disorders.

### RELATED APPLICATIONS

This application is related and claims priority to U.S. Provisional Application Ser. No. 60/788,565, filed Mar. 31, 2006, and U.S. Provisional Application Ser. No. 60/808,905, filed May 26, 2006. The contents of these applications are incorporated herein in their entireties by this reference.

### BACKGROUND

Thieno[2,3-d]pyrimidine derivatives were first introduced for the treatment of various depression disorders and higher dysfunctions of the brain. See, e.g., U.S. Pat. No. 4,695,568. For example, rats given exemplary thieno[2,3-d]pyrimidine derivatives exhibit up to about a 50% improvement in memory function in a passive avoidance response test. Since that time, it has also been shown that such compounds may also be useful, e.g., for the treatment of lower urinary tract disorders (see, e.g., U.S. Pat. No. 6,846,823), for the treatment of functional bowel disorders (see, e.g., U.S. Patent Application Publication No. 2005/0032780), as well as for the treatment of nausea, vomiting, and/or retching (see, e.g., U.S. Patent Application Publication No. 2004/0254171).

These previously disclosed thieno[2,3-d]pyrimidine derivatives, including 4-(2-fluorophenyl)-6-methyl-2-(piperazin-1-yl)thieno[2,3-d]pyrimidine hydrochloride, appear to show adequate solubility. However, higher solubility compounds may be desirable in therapeutic dosage forms.

### SUMMARY OF THE INVENTION

The present invention is based, at least in part, on the discovery of new salts of thieno[2,3-d]pyrimidine derivatives, e.g., 4-(2-fluorophenyl)-6-methyl-2-(piperazin-1-yl)thieno[2,3-d] pyrimidine salts, which have improved solubility properties and over existing thieno[2,3-d] pyrimidine salts. For example, salts of the present application have increased solubility, e.g., to provide formulations for easier or quicker oral release or to provide liquid formulations with increased dosages.

Accordingly, in one aspect, the present invention is directed to compounds which are thieno[2,3-d]pyrimidine derivatives including, but not limited to, highly soluble salts of Formula II: 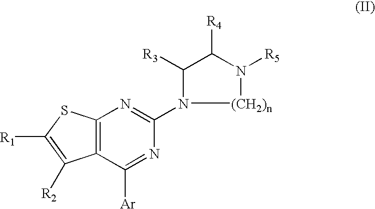
wherein

R₁ and R₂ are each independently hydrogen, halogen or a C₁-C₆ alkyl; or R₁ and R₂ are taken together with the carbons to which they are attached to form a 5 or 6 membered cycloalkylene group;

R₃ and R₄ are each independently hydrogen or a C₁-C₆ alkyl;

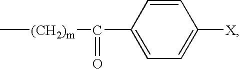

R₅ is hydrogen, C₁-C₆ alkyl,

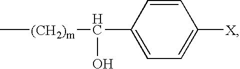

m is an integer from 1 to 3; X is a halogen; and R6 is a C₁-C₆ alkyl;

n is an integer of 2 or 3; and

Ar is a substituted or unsubstituted phenyl, 2-thienyl, or 3-thienyl group, wherein the solubility of said salt is greater than about 2.0 mg/ml in water as quantified by HPLC.

In some embodiments, R₁ is -CH₃. In other embodiments, R₂ is hydrogen. In some embodiments, R₃ is hydrogen. In other embodiments, R₄ is hydrogen. In still other embodiments, R₅ is hydrogen. In some embodiments, n is 2. In other embodiments, Ar is ortho-fluorophenyl.

In some embodiments, the compound is a 4-(2-fluorophenyl)-6-methyl-2-(piperazin-1-yl)thieno[2,3-d] pyrimidine salt.

In some embodiments, the salt is at least one salt selected from the group consisting of methane sulphonate, L-lactate, acetate and propionate.

In some embodiments, the solubility of the salt is greater than about 2.5 mg/ml in water, e.g., greater than about 5.0 mg/ml in water.

In some aspects, the present invention provides a pharmaceutical composition comprising at least one compound of any of the preceding claims and a pharmaceutically acceptable carrier.

In other aspects, the present invention is directed to a method for treating a gastrointestinal tract disorder and/or a genitourinary disorder in a subject. The method generally includes administering to the subject a composition comprising a therapeutically effective amount of any of the salts described herein, such that the gastrointestinal tract disorder and/or genitourinary disorder is treated. The gastrointestinal tract disorder or genitourinary disorder can be any of the gastrointestinal tract disorders or genitourinary disorders described herein. For example, gastrointestinal tract disorders or genitourinary disorders include, but are not limited to functional bowel disorders, irritable bowel syndrome, irritable bowel syndrome with diarrhea, chronic functional vomiting, overactive bladder or any combination thereof.

### BRIEF DESCRIPTION OF THE DRAWINGS

FIG. 1 is a table summarizing results of the salt selection studies with twelve exemplary salt formers.

### DETAILED DESCRIPTION OF THE INVENTION

The present invention is based, at least in part, on the discovery of new salts of thieno[2,3-d] pyrimidine derivatives, e.g., 4-(2-fluorophenyl)-6-methyl-2-(piperazin-1-yl)thieno[2,3-d] pyrimidine salts which have enhanced solubility profiles than currently available 4-(2-fluorophenyl)-6-methyl-2-(piperazin-1-yl)thieno[2,3-d]pyrimidine hydrochloride.

It is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation. However, so that the invention may be more readily understood, certain terms are first defined:

It is to be noted that the singular forms “a,” “an,” and “the” as used herein include “at least one” and “one or more” unless stated otherwise. Thus, for example, reference to “a pharmacologically acceptable carrier” includes mixtures of two or more carriers as well as a single carrier, and the like.

In the context of the present invention, a salt is considered to be “substantially pure” when less than about 6% (by weight), less than about 3%, or less than about 1% of the salt includes a different counterion. In addition, a substantially pure form of the salt may generally contain less than about 3% total impurities, and/or less than about 0.5% residual organic solvent. In other embodiments, the salts of the present invention contain more than trace amounts of water or residual organic solvent, e.g., in the case of a solvate, a hydrate or a hemihydrate.

Salts

In some aspects, the present invention is directed to salts of Formula I: 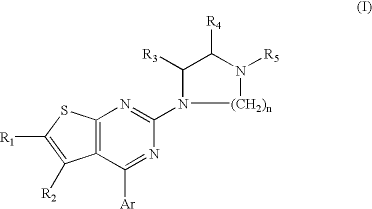
wherein
R₁ and R₂ are each independently hydrogen, halogen or a C₁-C₆ alkyl; or R₁ and R₂ are taken together with the carbons to which they are attached to form a 5 or 6 membered cycloalkylene group;
R₃ and R₄ are each independently hydrogen or a C₁-C₆ alkyl;
R₅ is hydrogen, C₁-C₆ alkyl, 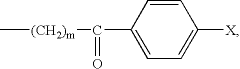 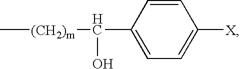
   or -C(O)—NH—R₆;
m is an integer from 1 to 3; X is a halogen; and R6 is a C₁-C₆ alkyl;
n is an integer of 2 or 3; and
Ar is a substituted or unsubstituted phenyl, 2-thienyl, or 3-thienyl group.

As used herein, the term “substituted” phenyl, 2-thienyl or 3-thienyl group refers to a phenyl, 2-thienyl or 3-thienyl group in which at least one of the hydrogen atoms available for substitution has been replaced with a group other than hydrogen (i.e., a substituent group). Multiple substituent groups can be present on the phenyl, 2-thienyl or 3-thienyl ring. When multiple substituents are present, the substituents can be the same or different and substitution can be at any of the substitutable sites on the ring. Substituent groups can be, for example, a halogen atom (fluorine, chlorine, bromine or iodine); an alkyl group, for example, a C₁-C₆ alkyl group such as a methyl, ethyl, propyl, butyl, pentyl or hexyl group; an alkoxy group, for example, a C₁-C₆ alkoxy group such as methoxy, ethoxy, propoxy, butoxy; a hydroxy group; a nitro group; an amino group; a cyano group; or an alkyl substituted amino group such as methylamino, ethylamino, dimethylamino or diethylamino group.

Alkyl group refers to a straight chain or branched alkyl group. C₁-C₆ alkyl group refers to a straight-chain or branched alkyl group having from one to six carbon atoms. For example, the C₁-C₆ alkyl group can be a strain-chain alkyl such as methyl, ethyl, propyl, etc. Alternatively, the alkyl group can be branched for example, an isopropyl or t-butyl group. Halogen refers to fluorine, chlorine, bromine or iodine.

In one embodiment, R₁ is a C₁-C₆ alkyl group and Ar is a substituted phenyl. In this embodiment, the phenyl group can be substituted with a halogen.

In another embodiment, n is 2, R₁ is a C₁-C₆ alkyl group and Ar is a substituted phenyl. In this embodiment, the phenyl group can be substituted with a halogen and the alkyl group of R₁ can be a methyl group.

In yet another embodiment, R₁ is a C₁-C₆ alkyl group or a halogen and Ar is an unsubstituted phenyl. Further, when R₁ is an alkyl group and Ar is an unsubstituted phenyl, R₂ can also be a hydrogen or a C₁-C₆ alkyl group.

In still another embodiment, n is 2, R₁ is a C₁-C₆ alkyl group and Ar is an unsubstituted phenyl. Further, when n is 2, R₁ is a C₁-C₆ alkyl group and Ar is an unsubstituted phenyl, R₂ can be hydrogen or a C₁-C₆ alkyl group.

In some embodiments, R₁ is -CH₃. In some embodiments, R₂ is hydrogen. In some embodiments, R₃ is hydrogen. In some embodiments, R₄ is hydrogen. In some embodiments, R₅ is hydrogen. In some embodiments, n is 2. In some embodiments, Ar is ortho-fluorophenyl.

In some embodiments, the compound is a 4-(2-fluorophenyl)-6-methyl-2-(piperazin-1-yl)thieno[2,3-d] pyrimidine salt. 4-(2-fluorophenyl)-6-methyl-2-(piperazin-1-yl)thieno[2,3-d] pyrimidine hydrochloride is known also as MCI-225. In some embodiments, the salts of the present invention are substantially pure.

In some embodiments, the salts of the present invention are highly soluble. As used herein, the term “highly soluble,” when used in reference to the salts of the present invention, refers to the salt being more soluble than conventionally available salts of the present invention, e.g., a 4-(2-fluorophenyl)-6-methyl-2-(piperazin-1-yl)thieno[2,3-d] pyrimidine salt being more soluble than 4-(2-fluorophenyl)-6-methyl-2-(piperazin-1-yl)thieno[2,3-d] pyrimidine hydrochloride. “Solubility” refers generally to the ability of a compound to dissolve, e.g., in water. Accordingly, in some embodiments, the salts of the present invention have a solubility of greater than about 2.0 mg/ml, greater than about 2.5 mg/ml, greater than about 3 mg/ml, 4 mg/ml, 5 mg/ml, 6 mg/ml, 7 mg/ml, 8 mg/ml, 9 mg/ml, 10 mg/ml or 20 mg/ml in water. It is to be understood that all values which fall in between the values listed are meant to be encompassed by the present invention. It is also to be understood that the solubility may be affected by the pH of the solution, and that enhanced solubility salts refer to salts that have enhanced solubility at any desired pH. In one embodiment, the solubility of the salt is greater than about 2.0 mg/ml in water as quantified by HPLC. In another embodiment, the solubility of the salt is greater than about 2.5 mg/ml in water as quantified by HPLC. In yet another embodiment, the solubility of the salt is greater than about 5.0 mg/ml in water as quantified by HPLC.

It is to be understood that the highly soluble salts of the present invention can be any pharmaceutically acceptable salts that exhibit high solubility as defined herein. As used herein, the term pharmaceutically acceptable salt refers to a salt of the administered compounds prepared from pharmaceutically acceptable non-toxic acids including inorganic acids, organic acids, solvates, hydrates, or clathrates thereof. Examples of such inorganic acids are hydrochloric, hydrobromic, hydroiodic, nitric, sulfuric, and phosphoric. Appropriate organic acids may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic (besylate), stearic, sulfanilic, alginic, galacturonic, and the like. In one embodiment, the salt includes, but is not limited to methane sulphonate, L-lactate, acetate and propionate.

Compounds of formula II can be synthesized using a number of methods including, but not limited to adding a salt former to a free-base form of a thieno[2,3-d] pyrimidine derivative. Salt formers include, but are not limited to hydrobromic acid, hydrochloric acid, 1,5-naphthalenedisulfonic acid, 1,5-naphthalenedisulfonic acid, sulphuric acid, 1,2-ethane disulfonic acid, cyclamic acid, p-toluene sulphonic acid, thiocyanic acid, methane sulphonic acid, dodecylsulfuric acid, naphthalene-2-sulphonic acid, benzene sulphonic acid, oxalic acid, 2-hydroxy ethanesulfonic acid, L-aspartic acid, maleic acid, phosphoric acid, ethane sulphonic acid, (1S)-camphor-10-sulphonic acid, L-glutamic acid, pamoic acid, 1-hydroxy-2-naphthoic acid, ketoglutaric acid (glutaric acid, 2-oxo), malonic acid, 2,5-dihydroxybenzoic acid (gentisic acid), salicylic acid, L-tartaric acid, fumaric acid, galactaric acid (mucic acid), citric acid, D-glucuronic acid, lactobionic acid, 4-amino salicylic acid, D-glucoheptonic acid, S-2-pyrrolidinon-5-carboxylic acid (pyroglutamic acid), DL-mandelic acid, D-mandelic acid, L-mandelic acid, L-malic acid, hippuric acid, formic acid, D-gluconic acid, glycolic acid, DL-lactic acid, L-lactic acid, oleic acid (z-octadecenoic), L-ascorbic acid, benzoic acid, succinic acid, 4-acetamidobenzoic acid, trans-cinnamic acid, adipic acid, (+)-camphoric acid, acetic acid, caproic acid, nicotinic acid, propionic acid, stearic acid (octadecanoic acid), undecylenic acid, caprylic acid (octanoic acid), orotic acid, and/or orotic acid monohydrate. Such salt formers can be used as a solid or liquid, or may be dissolved in a solvent, e.g., in an aqueous solution. Additionally, salt formers may also be used as salts, e.g., sodium or ammonium salts, of the acids listed herein. Compounds of formula II can also be synthesized using a number of methods known in the art.

Salt formers may be chosen for a variety of functions, e.g., modified solubility, safety, stability and/or efficacy. In some embodiments, salt formers are chosen based on pKa. In some embodiments, the salt former is chosen such that the pKa of the salt former is at least two units lower than that of the thieno[2,3-d]pyrimidine derivative.

It is understood that compounds of formula II can be identified, for example, by screening libraries or collections of molecules using suitable methods. Another source for the compounds of interest is combinatorial libraries which can comprise many structurally distinct molecular species. Combinatorial libraries can be used to identify lead compounds or to optimize a previously identified lead. Such libraries can be manufactured by well-known methods of combinatorial chemistry and screened by suitable methods.

In one embodiment, the compounds of the present invention can be used to treat MCI-225 responsive states. As used herein, the term “MCI-225 responsive states” include diseases, disorders, states and/or conditions that have been treated or are treatable with MCI-225 (4-(2-fluorophenyl)-6-methyl-2-(piperazin-1-yl)thieno[2,3-d] pyrimidine hydrochloride). Without wishing to be bound by any particular theory, it is believed that the compounds of the present invention will have the same or similar efficacy as MCI-225 in treating MCI-225 responsive states. In some embodiments, the compounds of the present invention have improved efficacy over MCI-225 in treating MCI-225 responsive states. In these methods of treatment and/or methods of use, the compounds of the present invention may also have at least one of the advantages described herein, e.g., photostability and/or solubility. Some methods for treatment and/or use are described in more detail hereinbelow.

Monoamine Neurotransmitters

In some embodiments, the compounds of the present invention affect the function of monoamine neurotransmitters. Monoamine neurotransmitters such as noradrenaline (also referred to as norepinephrine), serotonin (5-hydroxytryptamine, 5-HT) and dopamine are known and disturbances in these neurotransmitters have been indicated in many types of disorders, such as depression. These neurotransmitters travel from the terminal of a neuron across a small gap referred to as the synaptic cleft and bind to receptor molecules on the surface of a second neuron. This binding elicits intracellular changes that initiate or activate a response or change in the postsynaptic neuron. Inactivation occurs primarily by transport of the neurotransmitter back into the presynaptic neuron, which is referred to as reuptake. These neurons or neuroendocrine cells can be found both in the Central Nervous System (CNS) and in the Peripheral Nervous System (PNS). In the prior art, there are known compounds that affect the function of these monoamine neurotransmitters. There are known compounds that affect the function of a single monoamine neurotransmitter, as well as compounds that affect the function of a plurality of monoamine neurotransmitters. Some compounds interact with receptor sites more strongly and others interact more weakly. Moreover, some compounds are selective, while others are non-selective. All of these factors can affect the compound's ability to treat a targeted disease or condition. For compounds that affect the function of a plurality of monoamine neurotransmitters, the interplay between the function of the compound at the two receptor sites is not always entirely understood.

Without intending to be bound or limited by any theory, it is believed that in some embodiments, the compounds of the present invention affect the function of more than one monoamine neurotransmitter. It has been shown that MCI-225 is a dual NARI-5-HT₃ receptor agonist. As used herein, the term “dual NARI-5-HT₃ receptor agonist” refers to a compound that has some activity as both a noradrenaline reuptake inhibitor as well as a 5-HT₃ receptor agonist. It is also believed that MCI-225 interacts weakly at both the noradrenaline receptor site and the 5-HT₃ receptor site, and that MCI-225 is non-selective.

Accordingly, in some embodiments, the compounds of the present invention are dual NARI-5-HT₃ receptor agonists. In other embodiments, the compounds of the present invention are weak noradrenaline reuptake inhibitors as well as 5-HT₃ receptor agonists when compared with molecules that have purely NARI activity or purely 5-HT₃ receptor agonist activity. For example, the compounds of the present invention may exhibit weak activity at both the noradrenaline receptor site and the 5-HT₃ receptor site, rather than stronger activity at only one of the receptors. In still other embodiments, the dual NARI-5-HT₃ receptor agonists of the present invention are not selective. That is, in some embodiments, the compounds of the present invention do not have significantly more activity at the noradrenaline receptor than at the 5-HT₃ receptor or vice versa.

(I) Noradrenaline and Noradrenaline Reuptake Inhibitors:

Accordingly, in some embodiments, the compounds of the present invention are NorAdrenaline Reuptake Inhibitors. As used herein, the term NorAdrenaline Reuptake Inhibitor (NARI) refers to an agent (e.g., a molecule, a compound) which can inhibit noradrenaline transporter function. For example, a NARI can inhibit binding of a ligand of a noradrenaline transporter to said transporter and/or inhibit transport (e.g., uptake or reuptake of noradrenaline). As such, inhibition of the noradrenaline transport function in a subject can result in an increase in the concentration of physiologically active noradrenaline. It is understood that NorAdrenergic Reuptake Inhibitor and NorEpinephrine Reuptake Inhibitor (NERI) are synonymous with NorAdrenaline Reuptake Inhibitor (NARI).

As used herein, “noradrenaline transporter” refers to naturally occurring noradrenaline transporters (e.g., mammalian noradrenaline transporters (e.g., human (Homo sapiens) noradrenaline transporters, murine (e.g., rat, mouse) noradrenaline transporters)) and to proteins having an amino acid sequence which is the same as that of a corresponding naturally occurring noradrenaline transporter (e.g., recombinant proteins). The term includes naturally occurring variants, such as polymorphic or allelic variants and splice variants.

In certain embodiments, the NARI can inhibit the binding of a ligand (e.g., a natural ligand such as noradrenaline, or other ligand such as nisoxetine) to a noradrenaline transporter. In other embodiments, the NARI can bind to a noradrenaline transporter. For example, in one embodiment, the NARI can bind to a noradrenaline transporter, thereby inhibiting binding of a ligand to said transporter and inhibiting transport of said ligand. In another embodiment, the NARI can bind to a noradrenaline transporter, and thereby inhibit transport.

(II) Serotonin and 5-HT₃ Receptor Antagonists

In some embodiments, the compounds of the present invention are 5-HT₃ receptor antagonists. As used herein, the term 5-HT₃ receptor antagonist refers to an agent (e.g., a molecule, a compound) which can inhibit 5-HT₃ receptor function. For example, a 5-HT₃ receptor antagonist can inhibit binding of a ligand of a 5-HT₃ receptor to said receptor and/or inhibit a 5-HT₃ receptor-mediated response (e.g., reduce the ability of 5-HT₃ to evoke the von Bezold-Jarisch reflex).

As used herein, the term “5-HT₃ receptor” refers to ligand-gated ion channels that are extensively distributed, e.g., on enteric neurons in the human gastrointestinal tract, as well as other peripheral and central locations. Activation of these channels and the resulting neuronal depolarization have been found to affect the regulation of visceral pain, colonic transit and gastrointestinal secretions. Antagonism of the 5-HT₃ receptors has the potential to influence sensory and motor function in the gut. 5-HT₃ receptors can be naturally occurring receptors (e.g., mammalian 5-HT₃ receptors (e.g., human (Homo sapiens) 5-HT₃ receptors, murine (e.g., rat, mouse) 5-HT₃ receptors)) or proteins having an amino acid sequence which is the same as that of a corresponding naturally occurring 5-HT₃ receptor (e.g., recombinant proteins). The term includes naturally occurring variants, such as polymorphic or allelic variants and splice variants.

Recent animal studies have suggested that targeting 5-HT₃ receptors could offer additional treatments for lower urinary tract dysfunctions. For example, 5-HT₃ receptors mediate excitatory effects on sympathetic and somatic reflexes to increase outlet resistance. Moreover, 5-HT₃ receptors have also been shown to be involved in inhibition of the micturition reflex (Downie, J. W. (1999) Pharmacological manipulation of central micturition circuitry. Curr. Opin. SPNS Inves. Drugs 1:23). In fact, 5-HT₃ receptor inhibition has been shown to diminish 5-HT mediated contractions in rabbit detrusor (Khan, M. A. et al. (2000) Doxazosin modifies serotonin-mediated rabbit urinary bladder contraction. Potential clinical relevance. Urol. Res. 28:116).

In certain embodiments, the 5-HT₃ receptor antagonist can inhibit binding of a ligand (e.g., a natural ligand, such as serotonin (5-HT₃), or other ligand such as GR65630) to a 5-HT₃ receptor. In certain embodiments, the 5-HT₃ receptor antagonist can bind to a 5-HT₃ receptor. For example, in one embodiment, the 5-HT₃ receptor antagonist can bind to a 5-HT₃ receptor, thereby inhibiting the binding of a ligand to said receptor and a 5-HT₃ receptor-mediated response to ligand binding. In another embodiment, the 5-HT₃ receptor antagonist can bind to a 5-HT₃ receptor, and thereby inhibit a 5-HT₃ receptor-mediated response.

Methods of Treatment

Compounds and compositions of the present invention are useful for treating a number of gastrointestinal and/or genitourinary disorders in a subject. Accordingly, in some aspects, the present invention provides a method for treating a gastrointestinal disorder and/or a genitourinary disorder. The method includes administering at least one compound (e.g., one or more salts and/or polymorphs in a composition as described herein) such that the gastrointestinal and/or genitourinary disorder is treated. The gastrointestinal disorder can be any of the gastrointestinal disorders described herein. Furthermore, the genitourinary disorder can be any of the genitourinary disorder can be any of the genitourinary disorders described herein. For example, the disorder can be, but is not limited to, a functional bowel disorder, irritable bowel syndrome, irritable bowel syndrome with diarrhea, chronic functional vomiting, overactive bladder, or any combination thereof. It is to be understood that treatment of a disorder is meant to include treatment of at least one symptom of said disorder. For example, treatment of overactive bladder includes, but is not limited to, a lessening of urinary urgency.

“Treatment”, or “treating” as used herein, is defined as the application or administration of a therapeutic agent (e.g., a salt or polymorph of the present invention) to a subject who has a disorder, e.g., a gastrointestinal and/or genitourinary disorder as described herein, with the purpose to cure, heal, alleviate, delay, relieve, alter, remedy, ameliorate, improve or affect the disease or disorder, or symptoms of the disease or disorder. The term “treatment” or “treating” is also used herein in the context of administering agents prophylactically. The term “effective dose” or “effective dosage” is defined as an amount sufficient to achieve or at least partially achieve the desired effect. The term “therapeutically effective dose” is defined as an amount sufficient to cure or at least partially arrest the disease and its complications in a subject already suffering from the disease.

The term “subject,” as used herein, refers to animals such as mammals, including, but not limited to, humans, primates, cows, sheep, goats, horses, pigs, dogs, cats, rabbits, guinea pigs, rats, mice or other bovine, ovine, equine, canine, feline, rodent or murine species.

Gastrointestinal Disorders

In some embodiments, the compositions of the present invention are used to treat one or more gastrointestinal (GI) tract disorders. GI tract disorders may involve disturbances of the GI smooth muscle, epithelium, sensory afferent neurons, or central nervous system pathways. In spite of the uncertainty regarding whether central or peripheral mechanisms, or both, are involved in GI tract disorders, many proposed mechanisms implicate neurons and pathways that mediate visceral sensation.

GI tract disorders have been characterized as structural (or mucosal) GI tract disorders and non-structural (or non-mucosal) GI tract disorders. Structural disorders include inflammatory bowel disorders and non-inflammatory structural GI tract disorders. Non-structural disorders include a variety of disorders classified as functional GI tract disorders.

By “inflammatory bowel disorder” is intended any disorder primarily associated with inflammation of the small and/or large intestine, including but not limited to ulcerative colitis, Crohn's disease, ileitis, proctitis, celiac disease (or non-tropical sprue), enteropathy associated with seronegative arthropathies, microscopic or collagenous colitis, eosinophilic gastroenteritis, or pouchitis resulting after proctocolectomy, and post ileoanal anastomosis. Inflammatory bowel disorders include a group of disorders that can cause inflammation or ulceration of the GI tract. Ulcerative colitis and Crohn's disease are the most common types of inflammatory bowel disorders, although collagenous colitis, lymphocytic (microscopic) colitis, and other disorders have also been described.

“Crohn's disease” is used in its conventional sense to refer to gastrointestinal inflammation primarily of the small and large intestine, including disorders with fistulas or with extraintestinal manifestations, and encompasses all synonyms including regional enteritis, ileitis, and granulomatous ileocolitis.

“Proctitis” is used in its conventional sense to refer to inflammation of the rectal lining.

“Celiac disease” is used in its conventional sense to refer to any disorder primarily associated with altered sensititivity to gluten or gluten byproducts, with or without alterations in small bowel morphology (typically villus blunting) and encompasses all synonyms including celiac sprue and non-tropical sprue. Patients diagnosed with celiac disease may have symptomatic gluten intolerance with prominent diarrhea and abdominal pain or with minimal symptoms such as abdominal discomfort and associated dermatitis herpetiformis.

“Colitis” is used in its conventional sense to refer to inflammation of the large intestine.

“Ulcerative colitis” is used in its conventional sense to refer to inflammation and ulcers in the top layers of the lining of the large intestine and can be of any extent, including proctitis, proctosigmoiditis, left-sided colitis, or pan-colitis.

“Collagenous colitis” or “microscopic colitis” is used in its conventional sense to refer to an inflammatory disorder of unknown etiology with watery diarrhea as the leading symptom. A biopsy of the intestine typically demonstrates a thicker-than-normal layer of collagen (connective tissue) just beneath the inner surface of the colon (the epithelium) and/or inflammation of the epithelium and of the layer of connective tissue that lies beneath the epithelium. There is an association of arthritis with this disorder.

“Eosinophilic gastroenteritis” is used in its conventional sense to refer to a condition where a biopsy of the GI tract demonstrates infiltration with a type of white blood cell called eosinophils. There is no single cause of eosinophilic gastroenteritis and in many cases there is no known cause. Symptoms may include feeling full before finishing a meal, diarrhea, abdominal cramping or pain, nausea and vomiting. Asthma and allergies are sometimes related to the disorder.

“Pouchitis” is used in its conventional sense to refer to inflammation in a distal location of the intestine after a surgery on the intestine.

“Lymphocytic colitis” is used in its conventional sense to refer to inflammation of the large intestine without ulceration, and encompasses all synonyms including microscopic colitis.

The compounds of the present invention are useful in the treatment of ulcerative colitis. Ulcerative colitis is a chronic inflammatory disorder of unknown etiology afflicting the large intestine and, except when very severe, is limited to the bowel mucosa. The course of this disorder may be continuous or relapsing and may be mild or severe. Medical treatment primarily includes the use of salicylate derivatives, glucocorticosteroids such as prednisone or prednisone acetate and anti-metabolites dependent on the clinical state of the patient. Not only can such treatment can have a number of side effects, surgical removal of the colon to eliminate the disease may be needed in more severe, chronic cases.

The compounds of the present invention are also useful for in the treatment of Crohn's disease. Like ulcerative colitis, Crohn's disease (also known as regional enteritis, ileitis, or granulomatous ileocolitis) is a chronic inflammatory disorder of unknown etiology; however the location and pathology of the disease differ. Crohn's disease typically presents in either the small intestine, large intestine or the combination of the two locations and can cause inflammation deeper into the muscle and serosa located within the intestinal wall. The course of the disorder may be continuous or relapsing and may be mild or severe. Medical treatment includes the continuous use of salicylate derivatives, glucocorticosteroids, anti-metabolites, and administration of an anti-TNF antibody. Many Crohn's disease patients require intestinal surgery for a problem related to the disease, but unlike ulcerative colitis subsequent relapse is common.

Compounds of the present invention are also useful in the treatment of collagenous colitis and lymphocytic colitis. Collagenous colitis and lymphocytic colitis are idiopathic inflammatory disorders of the colon that cause watery diarrhea typically in middle-aged or older individuals. Lymphocytic colitis is distinguished from collagenous colitis by the absence of a thickened subepithelial collagenous layer. Bismuth in the form of Pepto-Bismol may be an effective treatment in some patients, although more severe cases may require the use of salicylate derivatives, antibiotics such as metronidazole, and glucocorticosteroids.

The term “non-structural GI tract disorder” or “non-mucosal GI tract disorder” refers to any GI tract disorder not related to structural or mucosal abnormalities of the GI tract, nor where there is evidence of a related metabolic disturbance, including but not limited to functional GI tract disorders.

Compounds of the present invention are also useful in the treatment of functional GI tract disorders. By “functional GI tract disorder” is intended any GI tract disorder associated with a disturbance of motor or sensory function in the absence of mucosal or structural damage or in the absence of a metabolic disorder. Functional GI tract disorders include functional dysphagia, non-ulcer dyspepsia, irritable bowel syndrome (IBS), slow-transit constipation and evacuation disorders. (Camilleri (2002) Gastrointestinal Motility Disorders, In WebMD Scientific American Medicine, edited by David C. Dale and Daniel D. Federman, New York, N.Y., WebMD). Functional GI tract disorders are characterized by presentation of abdominal-type symptoms without evidence of changes in metabolism or structural abnormalities.

In some embodiments, the functional GI tract disorder is a Functional Bowel Disorder. Functional Bowel Disorders (FBDs) are functional gastrointestinal disorders having symptoms attributable to the mid or lower gastrointestinal tract. FBDs can include, but are not limited to, Irritable Bowel Syndrome (IBS), functional abdominal bloating, functional constipation and functional diarrhea (see, for example, Thompson et al., Gut, 45 (Suppl. II):II43-1147 (1999)). Of these disorders, IBS alone accounts for up to about 3.5 million physician visits per year, and is the most common diagnosis made by gastroenterologists, accounting for about 25% of all patients (Camilleri and Choi, Aliment. Pharm. Ther., 11:3-15 (1997)).

In some embodiments, compounds and compositions of the present invention are useful in the treatment of IBS. At present, treatments for IBS include stress management, diet, and drugs. Such treatment, however, may have unwanted side effect or limited efficacy. Due to a lack of readily identifiable structural or biochemical abnormalities in IBS, the medical community has developed a consensus definition and criteria, known as the Rome II Criteria, to aid in diagnosis of IBS. Therefore, diagnosis of IBS is one of exclusion and is based on the observed symptoms in any given case. Diagnostic criteria, e.g., the Rome II criteria, for IBS, include at least 12 weeks in the preceding 12 months, which need not be consecutive, of abdominal pain or discomfort that has two of three features: (1) relieved with defecation; and/or (2) onset associated with a change in the frequency of stools; and/or (3) onset associated with a change in form (appearance) of stool.

Other symptoms, such as abnormal stool frequency (for research purposes “abnormal” can be defined as >3/day and <3/week); abnormal stool form (lumpy/hard or loose/watery stool); abnormal stool passage (straining, urgency, or feeling of incomplete evacuation); passage of mucus; bloating and/or feeling of abdominal distension, cumulatively support the diagnosis of IBS.

IBS can manifest in a number of varieties, including IBS constipation (IBS-c), IBS diarrhea (IBS-d) and IBS alternating (IBS-a). For example, IBS-c may be related to symptoms such as stool frequency of <3/week and straining, whereas IBS-d may be related to symptoms such as stool frequency of >3/day or loose/watery stool. IBS alternating is generally related to a manifestation of both IBS-c symptoms and IBS-d symptoms. Although the compounds of the present invention are useful for all manifestations, in some embodiments, the compounds of the present invention are useful in slowing functional bowel. Such compounds would be particularly effective for IBS-d.

Further, subjects with IBS can exhibit visceral hypersensitivity, the presence of which behavioral studies have shown is the most consistent abnormality in IBS. For example, patients and controls were evaluated for their pain thresholds in response to progressive distension of the sigmoid colon induced by a balloon. At the same volume of distension, the patients reported higher pain scores compared to controls. This finding has been reproduced in many studies and with the introduction of the barostat, a computerized distension device, the distension procedures have been standardized. Two concepts of visceral hypersensitivity, hyperalgesia and allodynia, have been introduced. More specifically, hyperalgesia refers to the situation in which normal visceral sensations are experienced at lower intraluminal volumes. While for a finding of allodynia, pain or discomfort is experienced at volumes usually producing normal internal sensations (see, for example, Mayer E. A. and Gebhart, G. F., Basic and Clinical Aspects of Chronic Abdominal Pain, Vol 9, l .sup.st ed. Amsterdam: Elsevier, 1993:3-28).

As such, IBS is a functional bowel disorder in which abdominal pain or discomfort is associated with defecation or a change in bowel habit. Therefore, IBS has elements of an intestinal motility disorder, a visceral sensation disorder, and a central nervous disorder. While the symptoms of IBS have a physiological basis, no physiological mechanism unique to IBS has been identified. In some cases, the same mechanisms that cause occasional abdominal discomfort in healthy individuals operate to produce the symptoms of IBS. The symptoms of IBS are therefore a product of quantitative differences in the motor reactivity of the intestinal tract, and increased sensitivity to stimuli or spontaneous contractions.

Compounds of the present invention are also useful in the treatment of non-ulcer dyspepsia. Non-ulcer dyspepsia (NUD) is another prominent example of a functional GI tract disorder with no established etiology. Symptoms related to NUD include nausea, vomiting, pain, early satiety, bloating and loss of appetite. Altered gastric emptying and increased gastric sensitivity and distress may contribute to NUD but do not completely explain its presentation. Treatments include behavioral therapy, psychotherapy, or administration of antidepressants, motility regulatory agents, antacids, H₂ -receptor antagonists, and prokinetics. However, many of these treatments have shown limited efficacy in many patients.

In addition to the structural/non-structural classification described above, GI tract disorders may also be sub-classified based upon anatomical, physiological, and other characteristics of different portions of the GI tract as described in Sleisenger and Fordtran's Gastrointestinal and Liver Disease, 6^{th} Ed. (W. B. Saunders Co. 1998); K. M. Sanders (1996) Gastroenterology, 111: 492-515; P. Holzer (1998) Gastroenterology,114: 823-839; and R. K. Montgomery et al. (1999) Gastroenterology, 116: 702-731. For example, acid peptic disorders are generally thought to arise from damage due to acidic and/or peptic activity of gastric secretions and may affect the esophagus, stomach, and duodenum. Acid peptic disorders include gastroesophageal reflux disease, peptic ulcers (both gastric and duodenal), erosive esophagitis and esophageal stricture. Zollinger-Ellison Syndrome may be considered an acid peptic disorder since it typically presents with multiple ulcers due to excessive acid secretion caused by an endocrine tumor. Treatments typically include gastric acid suppressive therapies, antibiotics, and surgery. In some patients, however, these therapies have proven ineffective. Therefore, the compounds of the present invention meet an existing need for treatments for acid peptic disorders.

Another sub-classification for GI tract disorders may be drawn between gastroesophageal and intestinal disorders based upon characteristics between different portions of the GI tract as disclosed in Sleisenger and Fordtran's Gastrointestinal and Liver Disease, 6^{th} Ed. (W. B. Saunders Co. 1998); K. M. Sanders (1996) Gastroenterology, 111: 492-515; P. Holzer (1998) Gastroenterology,114: 823-839; and R. K. Montgomery et al. (1999) Gastroenterology, 116: 702-731. Structural gastroesophageal disorders include disorders of the stomach and/or esophagus where there is no evidence of structural perturbations (including those observed in the mucosa) distal to the pylorus. Dyspepsia (chronic pain or discomfort centered in the upper abdomen) is a prominent feature of most structural gastroesophageal disorders but can also be observed in non-structural perturbations, and has been estimated to account for 2 to 5 percent of all general practice consultations. Structural gastroesophageal disorders include gastritis and gastric cancer. By contrast, structural intestinal tract disorders occur in both the small intestine (the duodenum, jejunum, and ileum) and in the large intestine. Structural intestinal tract disorders are characterized by structural changes in the mucosa or in the muscle layers of the intestine, and include non-peptic ulcers of the small intestine, malignancies, and diverticulosis. Non-peptic ulcers in the small intestine are typically related to administration of non-steroidal anti-inflammatory drugs. Diverticulosis is a disorder that rarely occurs in the small intestine and most commonly appears in the colon.

The compounds of the present invention are useful in the treatment of both gastroesophageal and intestinal disorders.

By “non-ulcer dyspepsia” is intended any disorder associated with any abdominal symptom after eating including nausea, vomiting, pain, early satiety, bloating and loss of appetite where no ulceration in present in the esophagus, stomach or duodenum. Altered gastric emptying, increased gastric sensitivity and distress are considered as factors in the development of non-ulcer dyspepsia.

By “irritable bowel syndrome” or “IBS” is intended any disorder associated with abdominal pain and/or abdominal discomfort and an alteration in bowel habit, and encompasses all symptoms including functional bowel, pylorospasm, nervous indigestion, spastic colon, spastic colitis, spastic bowel, intestinal neurosis, functional colitis, irritable colon, mucous colitis, laxative colitis, and functional dyspepsia.

As used herein, the term “functional abdominal bloating” refers generally to a group of functional bowel disorders which are dominated by a feeling of abdominal fullness or bloating and without sufficient criteria for another functional gastrointestinal disorder. Diagnostic criteria for functional abdominal bloating are at least 12 weeks, which need not be consecutive, in the preceding 12 months of: (1) feeling of abdominal fullness, bloating or visible distension; and (2) insufficient criteria for a diagnosis of functional dyspepsia, IBS, or other functional disorder.

As used herein, the term “functional constipation” refers generally to a group of functional disorders which present as persistent difficult, infrequent or seemingly incomplete defecation. The diagnostic criteria for functional constipation are at least 12 weeks, which need not be consecutive, in the preceding 12 months of two or more of: (1) straining in >¼ defecations; (2) lumpy or hard stools in >¼ defecations; (3) sensation of incomplete evacuation in >¼ defecations; (4) sensation of anorectal obstruction/blockade in >¼ defecation; (5) manual maneuvers to facilitate >¼ defecations (e.g., digital evacuation, support of the pelvic floor); and/or (6) <3 defecations/week. In some embodiments, loose stools are not present, and there are insufficient criteria for IBS.

As used herein, the term “functional diarrhea” refers to continuous or recurrent passage of loose (mushy) or watery stools without abdominal pain. The diagnostic criteria for functional diarrhea are at least 12 weeks, which need not be consecutive, in the preceding 12 months of: (1) Liquid (mushy) or watery stools; (2) Present >¾ of the time; and (3) No abdominal pain.

By “slow-transit constipation” is intended as a disorder with slowing of motility in the large intestine with a prolonged transit time through the organ.

By “evacuation disorders” is intended as any disorder where defecation occurs poorly and the patient is unable to expel stool.

By “acid peptic disorder” is intended any disorder associated with damage due to acidic and/or peptic activity of gastric secretions that affect the esophagus, stomach, and/or duodenum. Acid peptic disorders include gastroesophageal reflux disease, peptic ulcers (both gastric and duodenal), erosive esophagitis, esophageal strictures, and Zollinger-Ellison Syndrome.

GI tract disorders may divided between gastroesophageal and intestinal disorders based upon anatomical, physiological, and other characteristics of different portions of the GI tract as disclosed in Sleisenger and Fordtran's Gastrointestinal and Liver Disease, 6^{th} Ed. (W. B. Saunders Co. 1998); K. M. Sanders (1996) Gastroenterology, 111: 492-515; P. Holzer (1998) Gastroenterology, 114: 823-839; and R. K. Montgomery et al. (1999) Gastroenterology, 116: 702-731.

By “gastroesophageal” is intended all parts of the esophagus and stomach. By “gastroesophageal disorders” is intended any disorder involving the esophagus and/or duodenum. By “structural gastroesophageal disorder” is intended any disorder of the stomach and/or esophagus where there is no evidence of structural perturbations (including those observed in the mucosa) distal to the pylorus. Structural gastroesophageal disorders include gastric cancer and gastritis.

By “intestinal tract” is intended all parts of the duodenum, jejeunum, ileum and large intestine (or colon). By “intestinal tract disorder” is intended any disorder involving the duodenum, jejeunum, ileum, and large intestine (or colon). By “structural intestinal tract disorder” is intended any disorder involving the duodenum, jejeunum; ileum, and/or large intestine (or colon) where important mucosal and structural abnormalities are present or there is evidence of a related metabolic disturbance that is not an inflammatory bowel disorder or an acid peptic disorder. Structural intestinal disorders include ulcers typically related to medications such as non-steroidal anti-inflammatory drugs, malignancies, and diverticulosis.

By “small intestine” is intended all parts of the duodenum, jejunum, and ileum.

The term “duodenum” is used in its conventional sense to refer to that portion of the GI tract beginning at the pylorus and ending at the ligament of Treitz. The duodenum is divided into four parts. (See, e.g., Yamada (1999) Textbook of Gastroenterology 3d Ed., Lippincott Williams & Wilkins). The first part of the duodenum is also known as the superior portion of the duodenum, and begins at the pylorus, is about 5 cm long, and passes backward and upward beneath the liver to the neck of the gall bladder (the first 2-3 cm of which is the duodenal bulb). The second part of the duodenum is also known as the descending portion of the duodenum, and extends along the right margin of the head of the pancreas, and is approximately 7 to 10 cm in length. The third part of the duodenum is also known as the horizontal portion of the duodenum, and is where the duodenum passes from right to left across the spine, inclining upwards for about 5 to 8 cm. The fourth part of the duodenum is also known as the ascending portion of the duodenum and begins at the left of the vertebral column, ascends to the left of the aorta for 2 to 3 cm and ends at the ligament of Treitz.

In some embodiments, the GI disorder is a disorder associated with or exhibiting nausea, vomiting and/or retching, e.g., functional vomiting, chronic functional vomiting and/or cyclic vomiting syndrome. The act of vomiting, or emesis, can be described as the forceful expulsion of gastrointestinal contents through the mouth brought about by the descent of the diaphragm and powerful contractions of the abdominal muscles. Emesis is usually, but not always, preceded by nausea (the unpleasant feeling that one is about to vomit). Retching or dry heaves involves the same physiological mechanisms as vomiting, but occurs against a closed glottis, which prohibits the expulsion of gastric contents.

There are a number of groups of agents that have been used clinically for the treatment of emesis. These groups include: anticholinergics, antihistamines, phenothiazines, butyrophenones, cannabinoids, benzamides, glucocorticoids, benzodiazepines and 5-HT₃ receptor antagonists. In addition, tricyclic antidepressants have also been used on a limited basis. However, the undesirable side effects, such as dystonia and akathisia, sedation, anticholinergic effect and orthostatic hypotension, euphoria, dizziness, paranoid ideation, somnolence, extrapyramidal symptoms, diarrhea, perceptual disturbances, urinary incontinence, hypotension, amnesia, dry mouth, constipation, blurred vision, urinary retention, weight gain, hypertension and cardiac side effects, such as palpitations and arrhythmia continue to be associated with the use of such therapies, and are often are a significant drawback for this therapy.

Consequently, another embodiment of the present invention is a method for treating nausea, emesis/vomiting, retching or any combination thereof in a subject in need thereof comprising administering to said subject a therapeutically effective amount of any of the compounds described herein. In specific embodiments, the subject is a human.

Vomiting, nausea, retching or combinations thereof can be caused by a number of factors including, but not limited to, anesthetics, radiation, cancer chemotherapeutic agents, toxic agents, odors, medicines, for example, a scrotonin reuptake inhibitors (e.g., a selective serotonin reuptake inhibitors (SSRI)) or a dual serotonin-norepinephrine reuptake inhibitor (SNRI), analgesics such as morphine, antibiotics and antiparasitic agents, pregnancy, and motion. The language “chemotherapeutic agents,” as used herein, include, but are not limited to, for example, alkylating agents, e.g. cyclophosphamide, carmustine, lomustine, and chlorambucil; cytotoxic antibiotics, e.g. dactinomycin, doxorubicin, mitomycin-C, and bleomycin; antimetabolites, e.g. cytarabine, methotrexate, and 5-fluorouracil; vinca alkaloids, e.g. etoposide, vinblastine, and vincristine; and others such as cisplatin, dacarbazine, procarbazine, and hydroxyurea; and combinations thereof.

In the case of vomiting, nausea, retching caused by SSRI administration (e.g., daily SSRI administration), it is common for the adverse effects to diminish upon repeated administration of the drug, i.e., the patient becomes tolerant to the nausea-inducing effects of the SSRI. Accordingly, in certain embodiments, the invention features administration of a peripherally-restricted 5-HT3 receptor antagonist on an as needed basis, for example, prior to the induction of tolerance during a course of SSRI treatment.

Conditions which are associated with vertigo (e.g., Meniere's disease and vestibular neuronitis) can also cause nausea, vomiting, retching or any combination thereof. Headache, caused by, for example, migraine, increased intracranial pressure or cerebral vascular hemorrhage can also result in nausea, vomiting, retching or any combination thereof. In addition, certain maladies of the gastrointestinal (GI) tract, for example, cholecystitis, choledocholithiasis, intestinal obstruction, acute gastroenteritis, perforated viscus, dyspepsia resulting from, for example, gastroesophageal reflux disease, peptic ulcer disease, gastroparesis, gastric or esophageal neoplasms, infiltrative gastric disorders (e.g., Menetrier's syndrome, Crohn's disease, eosinophilic gastroenteritis, sarcoidosis and amyloidosis), gastric infections (e.g., CMV, fungal, TB and syphilis), parasites (e.g., Giardia lamblia and Strongyloides stercoralis), chronic gastric volvulus, chronic intestinal ischemia, altered gastric motility disorders and/or food intolerance or Zollinger-Ellison syndrome can result in vomiting, nausea, retching or any combination thereof. However, in some cases of vomiting, nausea, retching or any combination thereof, no etiology can be determined despite extensive diagnostic testing (e.g., cyclic vomiting syndrome).

In certain embodiments, vomiting is chronic functional vomiting (CFV). CFV is a chronic condition comprised of functional vomiting and cyclic vomiting syndrome, characterized by recurrent episodes of vomiting, nausea, and abdominal pain separated by symptom-free intervals. Accordingly, under Rome II Criteria, patients with CFV experience frequent episodes of vomiting occurring on at least three separate days in a week over three months, in conjunction with a history of three or more periods of intense, acute nausea and unremitting vomiting lasting hours to days, with intervening symptom-free intervals lasting weeks to months, in the absence of known medical and psychiatric causes. However, without wishing to be bound by theory, it is believed that CFV may be caused by the abnormal function (dysfunction) of the muscles or nerves controlling the organs of the middle and upper gastrointestinal (GI) tract.

Of significant clinical relevance is the nausea and vomiting resulting from the administration of general anesthetics (commonly referred to as, post-operative nausea and vomiting, PONV), chemotherapeutic agents and radiation therapy. In fact, the symptoms caused by the chemotherapeutic agents can be so severe that the patient refuses further treatment.

For example, three types of emesis are associated with the use of chemotherapeutic agents. The first type is acute emesis, which occurs within the first 24 hours of chemotherapy. The second type is delayed emesis which occurs 24 hours or more after chemotherapy administration. The third type is anticipatory emesis, which begins prior to the administration of chemotherapy, usually in patients whose emesis was poorly controlled during a previous chemotherapy cycle.

PONV is also an important patient problem and one that patients rate as the most distressing aspect of operative procedure, even above pain. Consequently, the need for an effective anti-emetic in this area is important. As a clinical problem PONV is troublesome and requires the presence of staff to ensure that vomitus is not regurgitated, resulting in very serious clinical sequelae. Furthermore, there are certain operative procedures where it is clinically important that patients do not vomit. For example, in ocular surgery where intra-cranial ocular pressure can increase to the extent that stitches are ruptured and the operative procedure is set back in terms of success to a marked degree.

Nausea, vomiting and retching are defined as acute when symptoms are present for less than a week. The causes of nausea, vomiting and retching of short duration are often separable from etiologies leading to more chronic symptoms. In contrast, nausea, vomiting and retching are defined as chronic when symptoms are present for over a week. For example, symptoms can be continuous or intermittent and last for months or years. In some embodiments, the compounds and compositions of the present invention are used to treat chronic functional vomiting.

In certain embodiments, the vomiting reflex may be triggered by stimulation of chemoreceptors in the upper GI tract and mechanoreceptors in the wall of the GI tract, which are activated by both contraction and distension of the gut as well as by physical damage. A coordinating center in the central nervous system controls the emetic response, and is located in the parvicellular reticular formation in the lateral medullary region of the brain. Afferent nerves to the vomiting center arise from abdominal splanchnic and vagal nerves, vestibulo-labyrinthine receptors, the cerebral cortex and the chemoreceptor trigger zone (CTZ). The CTZ lies adjacent to the area postrema and contains chemoreceptors that sample both blood and cerebrospinal fluid for noxious or toxic substances.

Direct links exist between the emetic center and the CTZ. In particular, the CTZ is exposed to emetic stimuli of endogenous origin (e.g., hormones), as well as to stimuli of exogenous origin, such as drugs. The efferent branches of cranial nerves V, VII and IX, as well as the vagus nerve and sympathetic pathways produce the complex coordinated set of muscular contractions, cardiovascular responses and reverse peristalsis that characterize vomiting.

Genitourinary Disorders
(a) Lower Urinary Tract Disorders

Lower urinary tract disorders affect the quality of life of millions of men and women in the United States every year. While the kidneys filter blood and produce urine, the lower urinary tract is concerned with storage and elimination of this waste liquid and includes all other parts of the urinary tract except the kidneys. Generally, the lower urinary tract includes the ureters, the urinary bladder, and the urethra. Disorders of the lower urinary tract include painful and non-painful overactive bladder, prostatitis and prostadynia, interstitial cystitis, benign prostatic hyperplasia, and, in spinal cord injured patients, spastic bladder and flaccid bladder.

Overactive bladder is a treatable medical condition that is estimated to affect 17 to 20 million people in the United States. Symptoms of overactive bladder include urinary frequency, urgency, nocturia (the disturbance of nighttime sleep because of the need to urinate) and urge incontinence (accidental loss of urine) due to a sudden and unstoppable need to urinate. As opposed to stress incontinence, in which loss of urine is associated with physical actions such as coughing, sneezing, exercising, or the like, urge incontinence is usually associated with an overactive detrusor muscle (the smooth muscle of the bladder which contracts and causes it to empty).

There is no single etiology for overactive bladder. Neurogenic overactive bladder (or neurogenic bladder) occurs as the result of neurological damage due to disorders such as stroke, Parkinson's disease, diabetes, multiple sclerosis, peripheral neuropathy, or spinal cord lesions. In these cases, the overactivity of the detrusor muscle is termed detrusor hyperreflexia. By contrast, non-neurogenic overactive bladder can result from non-neurological abnormalities including bladder stones, muscle disease, urinary tract infection or drug side effects.

Due to the enormous complexity of micturition (the act of urination) the exact mechanism causing overactive bladder is unknown. Overactive bladder may result from hypersensitivity of sensory neurons of the urinary bladder, arising from various factors including inflammatory conditions, hormonal imbalances, and prostate hypertrophy. Destruction of the sensory nerve fibers, either from a crushing injury to the sacral region of the spinal cord, or from a disease that causes damage to the dorsal root fibers as they enter the spinal cord may also lead to overactive bladder. In addition, damage to the spinal cord or brain stem causing interruption of transmitted signals may lead to abnormalities in micturition. Therefore, both peripheral and central mechanisms may be involved in mediating the altered activity in overactive bladder.

In spite of the uncertainty regarding whether central or peripheral mechanisms, or both, are involved in overactive bladder, many proposed mechanisms implicate neurons and pathways that mediate non-painful visceral sensation. Pain is the perception of an aversive or unpleasant sensation and may arise through a variety of proposed mechanisms. These mechanisms include activation of specialized sensory receptors that provide information about tissue damage (nociceptive pain), or through nerve damage from diseases such as diabetes, trauma or toxic doses of drugs (neuropathic pain) (See, e.g., A. I. Basbaum and T. M. Jessell (2000) The perception of pain. In Principles of Neural Science, 4th. ed.; Benevento et al. (2002) Physical Therapy Journal 82:601-12).

Current treatments for overactive bladder include medication, diet modification, programs in bladder training, electrical stimulation, and surgery. Currently, antimuscarinics (which are subtypes of the general class of anticholinergics) are the primary medication used for the treatment of overactive bladder. This treatment suffers from limited efficacy and side effects such as dry mouth, dry eyes, dry vagina, palpitations, drowsiness, and constipation, which have proven difficult for some individuals to tolerate.

Prostatitis and prostadynia are other lower urinary tract disorders that have been suggested to affect approximately 2-9% of the adult male population (Collins M M, et al., (1998) “How common is prostatitis? A national survey of physician visits,” Journal of Urology, 159: 1224-1228). Prostatitis is associated with an inflammation of the prostate, and may be subdivided into chronic bacterial prostatitis and chronic non-bacterial prostatitis. Chronic bacterial prostatitis is thought to arise from bacterial infection and is generally associated with such symptoms as inflammation of the prostate, the presence of white blood cells in prostatic fluid, and/or pain. Chronic non-bacterial prostatitis is an inflammatory and painful condition of unknown etiology characterized by excessive inflammatory cells in prostatic secretions despite a lack of documented urinary tract infections, and negative bacterial cultures of urine and prostatic secretions. Prostadynia (chronic pelvic pain syndrome) is a condition associated with the painful symptoms of chronic non-bacterial prostatitis without an inflammation of the prostate.

Currently, there are no established treatments for prostatitis and prostadynia. Antibiotics are often prescribed, but with little evidence of efficacy. COX-2 selective inhibitors and α-adrenergic blockers and have been suggested as treatments, but their efficacy has not been established. Hot sitz baths and anticholinergic drugs have also been employed to provide some symptomatic relief.

Interstitial cystitis is another lower urinary tract disorder of unknown etiology that predominantly affects young and middle-aged females, although men and children can also be affected. Symptoms of interstitial cystitis may include irritative voiding symptoms, urinary frequency, urgency, nocturia and suprapubic or pelvic pain related to and relieved by voiding. Many interstitial cystitis patients also experience headaches as well as gastrointestinal and skin problems. In some extreme cases, interstitial cystitis may also be associated with ulcers or scars of the bladder.

Past treatments for interstitial cystitis have included the administration of antihistamines, sodium pentosanpolysulfate, dimethylsulfoxide, steroids, tricyclic antidepressants and narcotic antagonists, although these methods have generally been unsuccessful (Sant, G. R. (1989) Interstitial cystitis: pathophysiology, clinical evaluation and treatment. Urology Annal 3: 171-196).

Benign prostatic hyperplasia (BPH) is a non-malignant enlargement of the prostate that is very common in men over 40 years of age. BPH is thought to be due to excessive cellular growth of both glandular and stromal elements of the prostate. Symptoms of BPH include urinary frequency, urge incontinence, nocturia, and reduced urinary force and speed of flow.

Invasive treatments for BPH include transurethral resection of the prostate, transurethral incision of the prostate, balloon dilation of the prostate, prostatic stents, microwave therapy, laser prostatectomy, transrectal high-intensity focused ultrasound therapy and transurethral needle ablation of the prostate. However, complications may arise through the use of some of these treatments, including retrograde ejaculation, impotence, postoperative urinary tract infection and some urinary incontinence. Non-invasive treatments for BPH include androgen deprivation therapy and the use of 5α-reductase inhibitors and α-adrenergic blockers. However, these treatments have proven only minimally to moderately effective for some patients.

Lower urinary tract disorders are particularly problematic for individuals suffering from spinal cord injury. After spinal cord injury, the kidneys continue to make urine, and urine can continue to flow through the ureters and urethra because they are the subject of involuntary neural and muscular control, with the exception of conditions where bladder to smooth muscle dyssenergia is present. By contrast, bladder and sphincter muscles are also subject to voluntary neural and muscular control, meaning that descending input from the brain through the spinal cord drives bladder and sphincter muscles to completely empty the bladder. Following spinal cord injury, such descending input may be disrupted such that individuals may no longer have voluntary control of their bladder and sphincter muscles. Spinal cord injuries can also disrupt sensory signals that ascend to the brain, preventing such individuals from being able to feel the urge to urinate when their bladder is full.

Following spinal cord injury, the bladder is usually affected in one of two ways. The first is a condition called “spastic” or “reflex” bladder, in which the bladder fills with urine and a reflex automatically triggers the bladder to empty. This usually occurs when the injury is above the T12 level. Individuals with spastic bladder are unable to determine when, or if, the bladder will empty. The second is “flaccid” or “non-reflex” bladder, in which the reflexes of the bladder muscles are absent or slowed. This usually occurs when the injury is below the T12/L1 level. Individuals with flaccid bladder may experience over-distended or stretched bladders and “reflux” of urine through the ureters into the kidneys. Treatment options for these disorders usually include intermittent catheterization, indwelling catheterization, or condom catheterization, but these methods are invasive and frequently inconvenient.

Urinary sphincter muscles may also be affected by spinal cord injuries, resulting in a condition known as “dyssynergia.” Dyssynergia involves an inability of urinary sphincter muscles to relax when the bladder contracts, including active contraction in response to bladder contraction, which prevents urine from flowing through the urethra and results in the incomplete emptying of the bladder and “reflux” of urine into the kidneys. Traditional treatments for dyssynergia include medications that have been somewhat inconsistent in their efficacy or surgery.

In addition to the lower urinary tract disorders described above, the related genitourinary tract disorders vulvodynia and vulvar vestibulitis have been etiologically and pathologically linked to such lower urinary tract disorders as interstitial cystitis (See Selo-Ojeme et al. (2002) Int. Urogynecol. J. Pelvic Floor Dysfunction 13: 261-2; Metts (2001) Am. Fam. Physician 64: 1199-206; Wesselmann (2001) World J Urol. 19: 180-5; Parsons et al. (2001) Obstet. Gynecol. 98: 127-32; Heim (2001) Am. Fam. Physician 63: 1535-44; Stewart et al. (1997) J. Reprod. Med. 42: 131-4; Fitzpatrick et al. (1993) Obstet. Gynecol. 81: 860-2). Vulvar vestibulitis syndrome (herein “vulvar vestibulitis”) is a subtype of vulvodynia. Vulvodynia is a complex gynecologic syndrome characterized by unexplained vulvar pain, sexual dysfunction, and psychological disability. Although the exact prevalence of vulvodynia is unknown, the condition is relatively common. It has been estimated that 1.5 million American women may suffer from some degree of vulvodynia.

The most common subtype of vulvodynia is vulvar vestibulitis (also called “focal vulvitis” and “vestibular adenitis”). Vulvar vestibulitis presents a constellation of symptoms involving and limited to the vulvar vestibule. The criteria for recognizing vulvar vestibulitis include: 1) pain on vestibular touch or attempted vaginal entry; 2) tenderness to Q-tip pressure localized within the vulvar vestibule; 3) physical findings confined to vestibular erythema of various degrees; and 4) an exclusion of other causes for vestibular erythema and tenderness, such as candidiasis (yeast infections) or herpes infections. Other symptoms include itching, swelling and excoriation.

The pain in vulvar vestibulitis may be described as sharp, burning, or a sensation of rawness. In severe cases, dyspareunia (recurrent or persistent genital pain associated with sexual intercourse) totally prohibits sexual intercourse. Pain may also be elicited on tampon insertion, biking, or wearing tight pants. The erythema may be diffuse or focal, and may be localized around the orifices of the vestibular glands or at the fourchette. In addition, patient symptoms may often include itching. Morbidities extend well beyond the local symptoms, with many women undergoing tremendous changes in psychosexual self-image, and can include profound adverse effects on marriages and other important relationships.

Vulvar vestibulitis may be acute or chronic. In one study, an arbitrary cutoff of three months of symptoms was used to distinguish between the acute and chronic forms (Marinoff and Turner, Am. J. Obstet. Gynecol. 165:1228-33, 1991). Most clinicians use an arbitrary cutoff of six months to distinguish between the acute and chronic forms. Some investigators have attempted to find a common histopathological aspect to vulvar vestibulitis, but have failed to do so (Pyka et al. (1988) Int. J Gynecol. Pathol. 7: 249-57).

The causes of vulvar vestibulitis are multifactorial. Known and suspected causes of the acute form include fungal or bacterial infection (e.g. Candida, Trichomonas), chemical irritants (e.g. soaps, douches, sprays), therapeutic agents (e.g. antiseptics, suppositories, creams, 5-fluorouracil methods (e.g. cryosurgery, laser treatment), and allergic drug reactions. In the acute form, treatment of the presumed cause may lead to rapid relief.

Vulvar vestibulitis may become chronic if the cause becomes persistent or recurrent and may persist long after all suspected causes have been treated. Many causes of chronic vulvar vestibulitis are of unknown etiology. Although no direct cause and effect relationship has been shown, it has been suggested that oxalates in the urine, altered vaginal pH, localized peripheral neuropathy, and subclinical viral infections can all contribute to the syndrome. A history of fungal infection is present in most patients who have vulvar vestibulitis, suggesting that recurrent yeast infections may somehow play a role in the initiation of the syndrome. It has been suggested that conditions such as recurrent candidiasis may lead to local changes in the vaginal immune system, including both Th1 and Th2 type responses (Fidel and Sobel, Clin. Microbiol. Reviews 9(3):335-48, 1996).

Because of its multiple causes, and its frequently unknown causes, vulvar vestibulitis can be very difficult to treat. The first-line therapy for vulvar vestibulitis is the treatment of its suspected causes. This includes the pharmacologic treatment of infections and the discontinued use of the irritants and therapeutic agents, local and systemic, that may contribute to the problem. Topical anesthetics, corticosteroids, and sex hormones may provide some symptomatic relief. Further treatments may include dietary modifications, physical therapy and biofeedback, use of topical, oral, or injected therapeutic agents, or surgery. Unfortunately, no single treatment works in all patients. Moreover, many of these approaches involve complex medical procedures, significant costs, and/or undesirable side effects.

By “lower urinary tract” is intended all parts of the urinary system except the kidneys. By “lower urinary tract disorder” is intended any disorder involving the lower urinary tract, including but not limited to overactive bladder, prostatitis, interstitial cystitis, benign prostatic hyperplasia, and spastic and flaccid bladder. By “non-painful lower urinary tract disorder” is intended any lower urinary tract disorder involving sensations or symptoms, including mild or general discomfort, that a patient subjectively describes as not producing or resulting in pain. By “painful lower urinary tract disorder” is intended any lower urinary tract disorder involving sensations or symptoms that a patient subjectively describes as producing or resulting in pain.

By “bladder disorder” is intended any condition involving the urinary bladder. By “non-painful bladder disorder” is intended any bladder disorder involving sensations or symptoms, including mild or general discomfort, that a patient subjectively describes as not producing or resulting in pain. By “painful bladder disorder” is intended any bladder disorder involving sensations or symptoms that a patient subjectively describes as producing or resulting in pain.

The language “overactive bladder (OAB)” refers to symptoms affecting the lower urinary tract which suggest detrusor muscle overactivity, in which the muscle contracts while the bladder is filling. Symptoms of OAB include urge to void, increased frequency of micturition or incontinence (involuntary loss of urine) and whether complete or episodic, where the loss of urine ranges from partial to total. By “painful overactive bladder” is intended any form of overactive bladder, as defined above, involving sensations or symptoms that a patient subjectively describes as producing or resulting in pain. By “non-painful overactive bladder” is intended any form of overactive bladder, as defined above, involving sensations or symptoms, including mild or general discomfort, that a patient subjectively describes as not producing or resulting in pain. Non-painful symptoms can include, but are not limited to, urinary urgency, incontinence, urge incontinence, stress incontinence, urinary frequency, and nocturia.

By “urinary urgency” is intended sudden strong urges to urinate with little or no chance to postpone the urination. By “incontinence” is meant the inability to control excretory functions, including urination (urinary incontinence). By “urge incontinence” or “urinary urge incontinence” is intended the involuntary loss of urine associated with an abrupt and strong desire to void. By “stress incontinence” or “urinary stress incontinence” is intended a medical condition in which urine leaks when a person coughs, sneezes, laughs, exercises, lifts heavy objects, or does anything that puts pressure on the bladder. By “urinary frequency” is intended urinating more frequently than the patient desires. As there is considerable interpersonal variation in the number of times in a day that an individual would normally expect to urinate, “more frequently than the patient desires” is further defined as a greater number of times per day than that patient's historical baseline. “Historical baseline” is further defined as the median number of times the patient urinated per day during a normal or desirable time period. By “nocturia” is intended being awakened from sleep to urinate more frequently than the patient desires. As used herein, “enuresis” refers to involuntary voiding of urine which can be complete or incomplete. Nocturnal enuresis refers to enuresis which occurs during sleep. Diurnal enuresis refers to enuresis which occurs while awake.

By “neurogenic bladder” or “neurogenic overactive bladder” is intended overactive bladder as described further herein that occurs as the result of neurological damage due to disorders including but not limited to stroke, Parkinson's disease, diabetes, multiple sclerosis, peripheral neuropathy, or spinal cord lesions.

By “detrusor hyperreflexia” is intended a condition characterized by uninhibited detrusor, wherein the patient has some sort of neurologic impairment. By “detrusor instability” or “unstable detrusor” is intended conditions where there is no neurologic abnormality.

By “prostatitis” is intended any type of disorder associated with an inflammation of the prostate, including chronic bacterial prostatitis and chronic non-bacterial prostatitis. By “non-painful prostatitis” is intended prostatitis involving sensations or symptoms, including mild or general discomfort, that a patient subjectively describes as not producing or resulting in pain. By “painful prostatitis” is intended prostatitis involving sensations or symptoms that a patient subjectively describes as producing or resulting in pain.

“Chronic bacterial prostatitis” is used in its conventional sense to refer to a disorder associated with symptoms that include inflammation of the prostate and positive bacterial cultures of urine and prostatic secretions. “Chronic non-bacterial prostatitis” is used in its conventional sense to refer to a disorder associated with symptoms that include inflammation of the prostate and negative bacterial cultures of urine and prostatic secretions. “Prostadynia” is used in its conventional sense to refer to a disorder generally associated with painful symptoms of chronic non-bacterial prostatitis as defined above, without inflammation of the prostate. “Interstitial cystitis” is used in its conventional sense to refer to a disorder associated with symptoms that include irritative voiding symptoms, urinary frequency, urgency, nocturia, and suprapubic or pelvic pain related to and relieved by voiding.

“Benign prostatic hyperplasia” is used in its conventional sense to refer to a disorder associated with benign enlargement of the prostate gland.

“Spastic bladder” or “reflex bladder” is used in its conventional sense to refer to a condition following spinal cord injury in which bladder emptying has become unpredictable.

“Flaccid bladder” or “non-reflex bladder” is used in its conventional sense to refer to a condition following spinal cord injury in which the reflexes of the bladder muscles are absent or slowed.

“Dyssynergia” is used in its conventional sense to refer to a condition following spinal cord injury in which patients characterized by an inability of urinary sphincter muscles to relax when the bladder contracts.

“Vulvodynia” is used in its conventional sense to refer to a condition characterized by gynecologic syndrome characterized by unexplained vulvar pain, sexual dysfunction, and psychological disability.

“Vulvar vestibulitis” (also known as “vulvar vestibulitis syndrome,” “focal vulvitis,” and “vestibular adenitis”) is used in its conventional sense to refer to a condition that is a subtype of vulvodynia characterized by: 1) pain on vestibular touch or attempted vaginal entry; 2) tenderness to Q-tip pressure localized within the vulvar vestibule; 3) physical findings confined to vestibular erythema of various degrees; and 4) an exclusion of other causes for vestibular erythema and tenderness, such as candidiasis (yeast infections) or herpes infections. Other symptoms may include itching, swelling and excoriation.

Additional Disorders

Additionally, the invention relates to methods of treating disorders which benefit from 5-HT₃ receptor antagonism. Some disorders have one or more significant peripheral components which benefit from 5-HT₃ receptor antagonism. Some disorders have both peripheral and CNS components which benefit from 5-HT₃ receptor antagonism, the compounds primarily treating the peripheral components. Some disorders have peripheral and/or CNS components and have CNS-mediated adverse effects or side effects. Disorders particularly suited for treatment according to the methodologies of the instant invention include those which benefit from 5-HT₃ receptor antagonism in the periphery (e.g., in the peripheral nervous system) and/or GI system, optionally having adverse or unwanted effects mediated by 5-HT₃ receptor activity in the CNS.

Accordingly, the invention additionally relates to a method treating pain, e.g., nociceptive or neoropathic pain, fibromyalgia and depressive conditions, obesity and weight gain, pre-menstrual syndrome, eating disorders, migraine, Parkinson's disease, stroke, schizophrenia, obsessive-compulsive disorder, fatigue, and any combination thereof. The method comprises administering to a subject in need of treatment thereof a therapeutically effective amount of a compound that has peripherally-restricted 5-HT₃ receptor antagonist activity.

Pharmaceutical Compositions and Modes of Administration

The present invention also encompasses pharmaceutical compositions including any of the compounds, e.g., the polymorphs or salts, described herein and a pharmaceutically acceptable carrier.

Pharmaceutically acceptable carriers include pharmaceutical diluents, excipients or carriers suitably selected with respect to the intended form of administration, and consistent with conventional pharmaceutical practices. For example, solid carriers/diluents include, but are not limited to, a gum, a starch (e.g., corn starch, pregelatinized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof.

Pharmaceutically acceptable carriers can be aqueous or non-aqueous solvents. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media.

In one embodiment, the pharmaceutical composition further comprises one or more additional therapeutic agents. The additional therapeutic agent can be any of the additional therapeutic agents described hereinbelow. Any further additional therapeutic agents useful for treating any of the diseases or disorders described herein may also be used in combination with the compositions of the present invention.

The compounds of the invention can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intraduodenal, intrathecal, subcutaneous, intramuscular, intradermal, intra-arterial, intravenous, inhalation, intrabronchial, intrapulmonary and topical administration. In one embodiment, the compositions of the present invention are formulated for oral administration.

Suitable compositions and dosage forms include tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. Further, those of ordinary skill in the art can readily deduce that suitable formulations involving these compositions and dosage forms, including those formulations as described elsewhere herein.

For example, for oral administration the compounds can be of the form of tablets or capsules prepared by conventional means with pharmaceutically acceptable excipients such as binding agents (e.g., polyvinylpyrrolidone, hydroxypropylcellulose or hydroxypropyl-methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets can be coated using suitable methods and coating materials such as OPADRY film coating systems available from Colorcon, West Point, Pa. (e.g., OPADRY OY Type, OY-C Type, Organic Enteric OY-P Type, Aqueous Enteric OY-A Type, OY-PM Type and OPADRY White, 32K18400). Liquid preparation for oral administration can be in the form of solutions, syrups or suspensions. The liquid preparations can be prepared by conventional means with pharmaceutically acceptable additives such as suspending agents (e.g., sorbitol syrup, methyl cellulose or hydrogenated edible fats); emulsifying agent (e.g. lecithin or acacia); non-aqueous vehicles (e.g., almond oil, oily esters or ethyl alcohol); and preservatives (e.g., methyl or propyl p-hydroxy benzoates or sorbic acid).

Tablets may be manufactured using standard tablet processing procedures and equipment. One method for forming tablets is by direct compression of a powdered, crystalline or granular composition containing the active agent(s), alone or in combination with one or more carriers, additives, or the like. As an alternative to direct compression, tablets can be prepared using wet-granulation or dry-granulation processes. Tablets may also be molded rather than compressed, starting with a moist or otherwise tractable material; however, compression and granulation techniques are preferred.

The dosage form may also be a capsule, in which case the active agent-containing composition may be encapsulated in the form of a liquid or solid (including particulates such as granules, beads, powders or pellets). Suitable capsules can be hard or soft, and are generally made of gelatin, starch, or a cellulosic material, with gelatin capsules preferred. Two-piece hard gelatin capsules are preferably sealed, such as with gelatin bands or the like. (See, for e.g., Remington: The Science and Practice of Pharmacy, supra), which describes materials and methods for preparing encapsulated pharmaceuticals. If the active agent-containing composition is present within the capsule in liquid form, a liquid carrier can be used to dissolve the active agent(s). The carrier should be compatible with the capsule material and all components of the pharmaceutical composition, and should be suitable for ingestion.

Compositions of the present invention may also be administered transmucosally. Transmucosal administration is carried out using any type of formulation or dosage unit suitable for application to mucosal tissue. For example, the selected active agent can be administered to the buccal mucosa in an adhesive tablet or patch, sublingually administered by placing a solid dosage form under the tongue, lingually administered by placing a solid dosage form on the tongue, administered nasally as droplets or a nasal spray, administered by inhalation of an aerosol formulation, and/or administered as an aerosol formulation, a non-aerosol liquid formulation (e.g., a suppository, ointment), or a dry powder, placed within or near the rectum or vagina (“transrectal,” “vaginal” and/or “perivaginal” formulations), or administered to the urethra (“transurethral” formulations) as a suppository, ointment, or the like.

Formulations suitable for other modes of administration, e.g., intravesical, intraduodenal, intrathecal, subcutaneous, intramuscular, intradermal, intra-arterial, intravenous, inhalation, intrabronchial, intrapulmonary and topical administration, are well known in the art and may be readily adapted for use with the compounds and compositions of the present invention.

Additional Dosage Formulations and Drug Delivery Systems

Further, the compounds for use in the method of the invention can be formulated in a sustained or otherwise controlled release preparation. For example, the compounds can be formulated with a suitable polymer or hydrophobic material which provides sustained and/or controlled release properties to the active agent compound. As such, the compounds for use the method of the invention can be administered in the form of microparticles for example, by injection or in the form of wafers or discs by implantation.

The formulations of the present invention can include, but are not limited to, short-term, rapid-offset, controlled, for example, sustained release, delayed release and pulsatile release formulations. For example, the compositions of the present invention may be used in controlled release systems developed by ALZA Corporation, Depomed Inc., and/or XenoPort Inc.

As used herein, the term “sustained release” refers to a drug formulation that provides for gradual release of a drug over an extended period of time, and that preferably, although not necessarily, results in substantially constant blood levels of a drug over an extended time period. The period of time can be as long as a month or more and should be a release which is longer that the same amount of agent administered in bolus form.

For sustained release, the compounds can be formulated with a suitable polymer or hydrophobic material which provides sustained release properties to the compounds. As such, the compounds for use the method of the invention can be administered in the form of microparticles for example, by injection or in the form of wafers or discs by implantation.

As used herein, the term “delayed release” refers to a drug formulation that provides for an initial release of the drug after some delay following drug administration and that preferably, although not necessarily, includes a delay of from about 10 minutes up to about 12 hours.

As used herein, the term “pulsatile release” refers to a drug formulation that provides release of the drug in such a way as to produce pulsed plasma profiles of the drug after drug administration.

As used herein, the term “immediate release” refers to a drug formulation that provides for release of the drug immediately after drug administration.

As used herein, “short-term” refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes after drug administration.

As used herein, “rapid-offset” refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes after drug administration.

Dosing

In some embodiments, the compounds of the present invention are administered in therapeutically effective amounts. The therapeutically effective amount or dose of a compound of the present invention will depend on the age, sex and weight of the subject, the current medical condition of the subject and the nature of the disorder being treated. The skilled artisan will be able to determine appropriate dosages depending on these and other factors.

As used herein, continuous dosing refers to the chronic administration of a selected active agent.

As used herein, as-needed dosing, also known as “pro re nata” “pm” dosing, and “on demand” dosing or administration is meant the administration of a therapeutically effective dose of the compound(s) at some time prior to commencement of an activity wherein suppression of a disorder would be desirable. Administration can be immediately prior to such an activity, including about 0 minutes, about 10 minutes, about 20 minutes, about 30 minutes, about 1 hour, about 2 hours, about 3 hours, about 4 hours, about 5 hours, about 6 hours, about 7 hours, about 8 hours, about 9 hours, or about 10 hours prior to such an activity, depending on the formulation.

In a particular embodiment, drug administration or dosing is on an as-needed basis, and does not involve chronic drug administration. With an immediate release dosage form, as-needed administration can involve drug administration immediately prior to commencement of an activity wherein suppression of symptoms of the disorder would be desirable, but will generally be in the range of from about 0 minutes to about 10 hours prior to such an activity, preferably in the range of from about 0 minutes to about 5 hours prior to such an activity, most preferably in the range of from about 0 minutes to about 3 hours prior to such an activity.

An exemplary suitable dose of the compounds of the present invention can be in the range of from about 0.001 mg to about 1000 mg per day, such as from about 0.05 mg to about 500 mg, for example, from about 0.03 mg to about 300 mg, such as from about 0.02 mg to about 200 mg per day. In a particular embodiment, a suitable dose of the compound can be in the range of from about 0.1 mg to about 50 mg per day, such as from about 0.5 mg to about 10 mg per, day such as about 0.5, 1, 2, 3, 4, 5, 6, 7, 8, 9, or 10 mg per day. Alternatively, the dose of the polymorph of the present invention can be greater than or equal to about 0.001 mg, about 0.005 mg, about 0.010 mg, about 0.020 mg, about 0.030 mg, about 0.040 mg, about 0.050 mg, about 0.100 mg, about 0.200 mg, about 0.300 mg, about 0.400 mg, about 0.500 mg, about 1 mg, about 1.5 mg, about 2.0 mg, about 2.5 mg, about 3.0 mg, about 3.5 mg, about 4.0 mg, about 4.5 mg, about 5 mg, about 10 mg, about 20 mg, about 30 mg, about 40 mg, about 50 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, or about 1000 mg. All values in between these values and ranges, e.g., 967 mg, 548 mg, 326 mg, 58.3 mg, 0.775 mg, 0.061 mg, are meant to be encompassed herein. All values in between these values and ranges may also be the upper or lower limits of a range, e.g., a particular dose may include a range of from 178 mg to 847 mg of the salt of the present invention.

The dose per day can be administered in a single dosage or in multiple dosages, for example from 1 to 4 or more times per day. When multiple dosages are used, the amount of each dosage can be the same or different. For example a dose of 1 mg per day can be administered as two 0.5 mg doses, with about a 12 hour interval between doses.

It is understood that the amount of compound dosed per day can be administered every day, every other day, every 2 days, every 3 days, every 4 days, every 5 days, etc. For example, with every other day administration, a 5 mg per day dose can be initiated on Monday with a first subsequent 5 mg per day dose administered on Wednesday, a second subsequent 5 mg per day dose administered on Friday, etc. It is also to be understood that the dosages do not have to be administered in any regular interval. That is, the first dose may be on day 1, the second dose on day 2, the third dose on day 5, the fourth on day 6, the fifth on day 12, etc.

The compounds for use in the method of the invention can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose. Dosing can also be on demand by the subject.

Each dosage can typically contain from about 0.001 mg to about 1000 mg, such as from about 0.05 mg to about 500 mg, for example, from about 0.03 mg to about 300 mg, such as about 0.02 mg to about 200 mg of the active ingredient.

Additional Therapeutic Agents

In one embodiment, the compositions of the present invention further comprise one or more additional therapeutic agents.

Additional therapeutic agents suitable for use in the methods and pharmaceutical compositions described herein include, but is not limited to, an antimuscarinic (e.g., oxybutynin, DITROPAN, tolterodine, flavoxate, propiverine, trospium); a muscosal surface protectant (e.g., ELMIRON); an antihistamine (e.g., hydroxyzine hydrochloride or pamoate); an anticonvulsant (e.g., NEURONTIN and KLONOPIN); a muscle relaxant (e.g., VALIUM); a bladder antispasmodic (e.g., URIMAX); a tricyclic antidepressant (e.g., imipramine); a nitric oxide donor (e.g., nitroprusside), a β₃-adrenergic receptor agonist, a bradykinin receptor antagonist, a neurokinin receptor antagonist, a sodium channel modulator, such as TTX-R sodium channel modulator and/or activity dependent sodium channel modulator and a Cav2.2 subunit calcium channel modulator. Such agents are known in the art and are generally listed in U.S. Pat. No. 6,846,823. In some embodiments, additional therapeutic agents are useful for treating the disorder of interest. In some embodiments, additional therapeutic agents do not diminish the effects of the primary agent(s) and/or potentiates the effect of the primary agent(s).

An additional therapeutic agent suitable for use in the methods and pharmaceutical compositions described herein, can be, but is not limited to, for example: an antispasmodic agent, such as an anticholinergic drug (e.g., dicyclomine, hyoscyamine, scopolamine, and cimetropium); a smooth muscle relaxant (e.g., mebeverine); a calcium blocker (e.g., verapamil, nifedipine, octylonium bromide, peppermint oil and pinaverium bromide); an antidiarrheal agent (e.g., loperamide and dipehnoxylate); a stool bulking agent (e.g., psyllium, polycarbophil); an antiafferent agent (e.g., octreotide and fedotozine); a prokinetic agent, such as a dopamine antagonist (e.g., domperidone and metoclopramide) or a 5-HT₄ antagonist (e.g., cisapride); a psychotropic agent, an antihistamine (e.g., dimenhydrinate and diphenhydramine); a phenothiazine (e.g., prochlorperazine and chlorpromazine); a butyrophenone (haloperidol and droperidol); a cannabinoid (e.g., tetrahydrocannabinol and nabilone); a benzamide (e.g., metoclopramide, cisapride and trimethobenzamide); a glucocorticoid (e.g., dexamethasone and methylprednisolone); a benzodiazepine (e.g., lorazepaam); or any combination thereof.

In some embodiments, use of an additional therapeutic agent in combination with the compounds of the present invention can result in less of any of the agent(s) and/or less of the additional agent being needed to achieve therapeutic efficacy. In some instances, use of less of an agent can be advantageous in that it provides a reduction in undesirable side effects.

In practicing the methods of the invention, coadministration refers to administration of a compound of the invention, e.g., a polymorph or salt of the invention, with an additional compound to treat a disorder. Coadministration encompasses administration of the first and second amounts of the compounds of the coadministration in an essentially simultaneous manner, such as in a single pharmaceutical composition, for example, capsule or tablet having a fixed ratio of first and second amounts, or in multiple, separate capsules or tablets for each. In addition, such coadministration also encompasses use of each compound in a sequential manner in either order. In some embodiments, the compounds are administered sufficiently close in time to have the desired therapeutic effect.

Kits

The invention further includes a kit for treating a disease or disorder of the present invention. The kit comprises at least one compound of the present invention and an instruction insert for administering the compound according to the method of the invention. In other embodiments of the kits, the instructional insert further includes instructions for administration with an additional therapeutic agent as described herein.

It is understood that in practicing the method or using a kit of the present invention that administration encompasses administration by different individuals (e.g., the subject, physicians or other medical professionals) administering the same or different compounds.

Pharmacological Methods

Acute Models: Dilute Acetic Acid Model and Protamine Sulfate/Phvsiological Urinary Potassium Model

The acute models described below provide methods for evaluating active agents in the treatment of overactive bladder. Briefly, the models provide a method for reducing the bladder capacity of test animals by infusing either protamine sulfate and potassium chloride (See, Chuang, Y. C. et al., Urology 61(3): 664-670 (2003)) or dilute acetic acid (See, Sasaki, K. et al., J. Urol. 168(3): 1259-1264 (2002)) into the bladder. The infusates cause irritation of the bladder and a reduction in bladder capacity by selectively activating bladder afferent fibers, such as C-fiber afferents. Following irritation of the bladder, an active agent (drug) can be administered and the ability of the active agent to reverse (partially or totally) the reduction in bladder capacity resulting from the irritation, can be determined. Substances which reverse the reduction in bladder capacity can be used in the treatment of overactive bladder.
(a) Animal Preparation for Acute Models:

Female rats (250-275 g BW) are anesthetized with urethane (1.2 g/kg) and a saline-filled jugular catheter (PE-50) is inserted for intravenous drug administration and a heparinized (100 units/ml) saline-filled carotid catheter (PE-50) is inserted for blood pressure monitoring. Via a midline abdominal incision from xyphoid to navel, a PE-50 catheter is inserted into the bladder dome for bladder filling and pressure recording. The abdominal cavity is moistened with saline and closed by covering with a thin plastic sheet in order to maintain access to the bladder for filling cystometry emptying purposes. Fine silver or stainless steel wire electrodes are inserted into the external urethral sphincter (EUS) percutaneously for electromyography (EMG).
(b) Dilute Acetic Acid Model:

Saline and all subsequent infusates are continuously infused at a rate of about 0.055 ml/min via the bladder filling catheter for 30-60 minutes to obtain a baseline of lower urinary tract activity (continuous cystometry; CMG). Bladder pressure traces act as direct measures of bladder and urethral outlet activity, and EUS-EMG phasic firing and voiding act as indirect measures of lower urinary tract activity during continuous transvesical cystometry. Following the control period, a 0.25% acetic acid solution in saline (AA) is infused into the bladder to induce bladder irritation. Following 30 minutes of AA infusion, 3 vehicle injections are made at 20 minute intervals to determine vehicle effects, if any. Subsequently, increasing doses of a selected active agent are administered intravenously at 30 minute intervals in order to construct a cumulative dose-response relationship. At the end of the control saline cystometry period, the third vehicle injection, and 20 minutes following each subsequent treatment, the infusion pump is stopped, the bladder is emptied by fluid withdrawal via the infusion catheter and a single filling cystometrogram is performed at the same flow rate in order to determine changes in bladder capacity caused by the irritation protocol and subsequent drug administration. In this acute model, C-fiber afferent pathways within the bladder are selectively activated.
(c) Protamine Sulfate/Physiological Urinary Potassium Model:

Saline and all subsequent infusates are continuously infused at a rate of about 0.055 ml/min via the bladder filling catheter for about 30-60 minutes to obtain a baseline of lower urinary tract activity (continuous cystometry; CMG). Bladder pressure traces act as direct measures of bladder and urethral outlet activity, and EUS-EMG phasic firing and voiding act as indirect measures of lower urinary tract activity during continuous transvesical cystometry. Following the control period, a 10 mg/mL protamine sulfate (PS) in saline solution is infused for about 30 minutes in order to permeabilize the urothelial diffusion barrier. After PS treatment, the infusate is switched to 300 mM KCl in saline to induce bladder irritation. Once a stable level of lower urinary tract hyperactivity is established (20-30 minutes), 3 vehicle injections are made at about 30 minute intervals to assess the effects of the vehicle. Subsequently, increasing doses of a selected active agent are administered intravenously at about 30 minute intervals in order to construct a cumulative dose-response relationship. At the end of the control saline cystometry period, the third vehicle injection, and 20 minutes following each subsequent treatment, the infusion pump is stopped, the bladder is emptied by fluid withdrawal via the infusion catheter and a single filling cystometrogram is performed at the same flow rate in order to determine changes in bladder capacity caused by the irritation protocol and subsequent drug administration. This model acutely activates bladder afferent fibers, including, C-fiber afferents.

Chronic Model: Chronic Spinal Cord Injury Model

The following is a model of neurogenic bladder, in which C-fiber afferents are chronically activated as a result of spinal cord injury (See, Yoshiyama, M. et al., Urology 54(5): 929-933 (1999)). Following spinal cord injury an active agent (drug) can be administered and the ability of the active agent to reverse (partially or totally) the reduction in bladder capacity resulting from spinal cord injury can be determined. Substances which reverse the reduction in bladder capacity can be used in the treatment of overactive bladder, for example, neurogenic bladder.
(a) Animal Preparation for Chronic Model:

Female Sprague-Dawley rats (Charles River, 250-300 g) are anesthetized with isofluorane (4%) and a laminectomy is performed at the T9-10 spinal level. The spinal cord is transected and the intervening space filled with Gelfoam. The overlying muscle layers and skin are sequentially closed with suture, and the animals are treated with antibiotic (100 mg/kg ampicillin s.c.). Residual urine is expressed prior to returning the animals to their home cages, and thereafter 3 times daily until terminal experimentation four weeks later. On the day of the experiment, the animals are anesthetized with isofluorane (4%) and a jugular catheter (PE10) is inserted for access to the systemic circulation and tunneled subcutaneously to exit through the midscapular region. Via a midline abdominal incision, a PE50 catheter with a fire-flared tip is inserted into the dome of the bladder through a small cystotomy and secured by ligation for bladder filling and pressure recording. Small diameter (75 .mu.m) stainless steel wires are inserted percutaneously into the external urethral sphincter (EUS) for electromyography (EMG). The abdominal wall and the overlying skin of the neck and abdomen are closed with suture and the animal is mounted in a Ballman-type restraint cage. A water bottle is positioned within easy reach of the animal's mouth for ad libitum access to water. The bladder catheter is hooked up to the perfusion pump and pressure transducer, and the EUS-EMG electrodes to their amplifier. Following a 30 minute recovery from anesthesia and acclimatization, normal saline is infused at a constant rate (0.100-0.150 ml/min) for control cystometric recording.
(b) Chronic Spinal Cord Injury Model:

Following a 60-90 minute control period of normal saline infusion (0.100-0.150 ml/min) to collect baseline continuous open cystometric data, the pump is turned off, the bladder is emptied, the pump turned back on, and bladder capacity is estimated by a filling cystometrogram. At 3×20-30 minute intervals, vehicle is administered intravenously in order to ascertain vehicle effects on bladder activity. Following the third vehicle control, bladder capacity is again estimated as described above. Subsequently, a cumulative dose-response is performed with the agent of choice. Bladder capacity is measured 20 minutes following each dose. This is a model of neurogenic bladder, in which C-fiber afferents are chronically activated.

Anti-Emetic Effects

The activity of compounds as anti-emetics can be demonstrated by any suitable model. For example, the extent to which compounds can reduce the latency or the number of retches and/or vomits induced by emetogens (e.g., cisplatin which is a typically used emetogenic trigger in suitable animal models) in, for example, the dog (e.g., beagles), the piglet or in the ferret can be assessed. For example, suitable methods are described in Tatersall et al. and Bountra et al., European Journal of Pharmacology, 250: (1993) R5 and 249:(1993) R3-R4 and Milano et al., J. Pharmacol. Exp. Ther., 274(2): 951-961 (1995).

In addition, the general method described by Florezyk et al., Cancer Treatment Report, 66(1): 187-9, (1982)) and summarized below, can also be used to assess effect of a test compound on emesis in the ferret.

Briefly, both the test compound and cisplatin are prepared and administered. The cisplatin is a representative emetogenic trigger for vomiting.
a) Control—Without Test Agent

Emesis is induced in groups of 6 male ferrets weighing about 2 kg, by intravenous administration of cisplatin at a suitable dose (e.g., 10 mg/kg). The onset of emesis is noted. Over a period of 2 hours the number of vomits/retches (episodes) is recorded. Behavioral changes characteristic of emesis are also noted.
b) With Test Compound

The test compound is administered to groups of 6 male ferrets weighing about 2 kg, by intravenous administration at suitable doses immediately prior to administration of cisplatin as described above. The animals are observed for 3 hours.

The emetic response seen in drug tested and control animals can then be compared to assess antiemetic properties of the test compound.

Distension Models A variety of assays can be used to assess visceromotor and pain responses to rectal distension. See, for example, Gunter et al., Physiol. Behav., 69(3): 379-82 (2000), Depoortere et al., J. Pharmacol. and Exp. Ther., 294(3): 983-990 (2000), Morteau et al., Fund. Clin. Pharmacol., 8(6): 553-62 (1994), Gibson et al., Gastroenterology (Suppl. 1), 120(5): A19-A20 (2001) and Gschossmann et al., Eur. J. Gastro. Hepat., 14(10): 1067-72 (2002) the entire contents of which are each incorporated herein by reference.

Visceral Pain

Visceral pain can lead to visceral reactions which can manifest themselves as, for example, contractions of the abdominal muscles. The number of contractions of the abdominal muscles occurring after a mechanical pain stimulus produced by distending the large intestine can thus be a measurement for determining visceral sensitivity to pain.

The inhibiting action of a test agent on distension-induced contractions can be tested in rats. The distension of the large intestine with an introduced balloon can be used as the stimulus; the contraction of the abdominal muscles can be measured as the response.

For example, one hour after sensitizing of the large intestine by instillation of a weak acetic acid solution, a latex balloon is introduced and inflated sequentially in a stepwise fashion to about 50-100 mbar for about 5-10 minutes. Pressure values can also be expressed as cm H₂0 at 4° C. (mbar X 1.01973=cm H₂0 at 4° C.). During this time, the contractions of the abdominal muscles are counted. About 20 minutes after subcutaneous administration of the test agent, this measurement is repeated. The action of the test agent is calculated as a percentage reduction in the counted contractions compared with the control (i.e., non-sensitized rats).

Gastrointestinal (GI) Motility Model

The investigation of gastrointestinal motility can be based on either the in vivo recording of mechanical or electrical events associated intestinal muscle contractions in whole animals or the activity of isolated gastrointestinal intestinal muscle preparations recorded in vitro in organ baths (see, for example, Yaun et al., Br. J. Pharmacol., 112(4):1095-1 100 (1994), Jin et al., J. Pharm. Exp. Ther., 288(1): 93-97 (1999) and Venkova et al., J. Pharm. Exp. Ther., 300(3):1046-1052 (2002)). The in vivo recordings, especially in conscious freely moving animals, have the advantage of characterizing motility patterns and propulsive activity that are directly relevant to the motor function of the GI tract. In comparison, in vitro studies provide data about the mechanisms and site of action of agents directly affecting contractile activity and are a classic tool to distinguish between effects on the circular and/or longitudinal intestinal smooth muscle layers.

(a) In Vivo
(i) Colonic Contractility

Ambulatory telemetric motility recordings provide a suitable way to investigate intestinal motility in conscious animals during long-lasting time periods. Telemetric recording of colonic motility has been introduced to study propagating contractile activity in the unprepared colon of conscious freely moving animals. Yucatan mini-pigs, present an excellent animal model for motility investigations, based on the anatomical and functional similarities between the gastrointestinal tract in the human and the mini-pig. To be prepared for studies of colonic motility, young mini-pigs undergo a surgical procedure to establish a permanent chronic cecal fistula.

During an experimental trial, the animals are housed in an animal facility under controlled conditions and receive a standard diet with water available ad libitum. Telemetric recording of colonic motility in a segment of proximal colon in the mini-pig is carried out for approximately one week (McRorie et al., Dig. Dis. Sci. 43: 957-963 (1998); Kuge et al., Dig. Dis. Sci. 47: 2651-6 (2002)). The data obtained in each recording session can be used to define the mean amplitude and the total number of propagating contractions, the number of high and low velocity propagating contractions, the number of long and short duration propagating contractions and to estimate the relative shares of each type contractions as % of total contractile activity. A summarized motility index (MI), characterizing colonic contractile activity, can be calculated using the following equation: 2 MI=# of contractions/24 hr. X area under the pressure peak 24 hr.
(ii) Colonic Motility

Female rats are administered, TNBS in ethanol or saline (control), intracolonically. The catheter tip is positioned between 2 and 6 cm from the anal verge (n=6/group). Three days following TNBS administration, the animals are food restricted overnight and on the following morning are anesthetized with urethane and are instrumented for physiological/pharmacological experimentation.

A ventral incision is made on the ventral surface of the neck, a jugular catheter is inserted and secured with ligatures, and the skin wound is closed with suture. An intra-colonic balloon-tipped catheter fashioned from condom reservoir tip and tubing is inserted anally and positioned with the balloon at approximately 4 cm from the anal verge. Connection via 3-way stopcock to a syringe pump and pressure transducer allows for simultaneous balloon volume adjustment and pressure recording. Fine wire electrodes are inserted into the external anal sphincter (EAS) and the abdominal wall musculature to permit electromyographic (EMG) recording. With this preparation, intra-colonic pressure, colonic motility, colonic sensory thresholds via abdominal EMG firing, and EAS firing frequency and amplitude is quantified in both control and irritated animals.

Following a control period of about 1 hour at a balloon volume of about 0.025 ml to establish baseline colonic motility and associated non-noxious viscero-somatic reflex measurements, three consecutive escalating ramps of stepwise or continuous balloon inflation are conducted. Following the completion of each volume ramp, the balloons are deflated for 30 minutes for recovery and collection of additional colonic motility measurements. EMG and colonic pressure responses to balloon inflation are measured and analyzed as sensitivities to colorectal distension (CRD). Administration of pharmacological agents is conducted in an escalating dose-response protocol and begins following the last control CRD balloon deflation.
(b) In Vitro

Recordings of contractile activity of isolated smooth muscle preparations can be used to study selected aspects of muscle function under conditions where the influence of “external” factors (circulating hormones etc.) is removed, while the muscle itself retains its in vivo capacity.

Studies are performed using smooth muscle strips (or whole intestinal segments) mounted vertically in organ baths with one end fixed and the other attached to isometric force transducers. The muscles are continuously bathed in modified Krebs bicarbonate buffer, maintained at 37° C. and aerated with 95% O₂ and 5% CO₂. The tissues are allowed to equilibrate at initial length (Lᵢ—at which tension is zero) for approximately 5 minutes, and then are gradually stretched by small force increments to optimal length (Lₒ—the length at which maximal active tension is generated in response to an agonist). Experiments should be performed at Lo to provide standardized spontaneous activity and pharmacological responses. The most commonly used recording procedures involve isometric transducers attached to an appropriate recording device. Mechanical responses to stimulation of enteric nerve terminals can be studied in organ baths supplied with pairs of platinum electrodes connected to a physiological electrical stimulator. Isolated smooth muscle preparations can be used also to study length-tension relationships, which provide characteristics of the active and passive properties of the smooth muscle.

Clinical Evaluation—Trial Design for Phase II

The phase II is a dose ranging study that is randomized, double blind placebo controlled parallel group multicenter study in adult (age 18 and over) men and women. In some studies, the patient population can be limited to women.

This is a 2-week run in study with a 4 or 12-week active treatment phase followed by a 2-week minimum follow-up phase to assess treatment of drug in patients with IBS. Subjects will need to fulfill Rome 11-type criteria for IBS with at least 6 months of symptoms. Subjects are ambulatory outpatients, have evidence of a recent examination of the large intestine, with no evidence of other serious medical conditions including inflammatory bowel disease.

There are three phases to the study. There is a 2-week screening period to confirm the symptomatology and record changes in bowel habit. Randomization of all subjects that continue to be eligible will be made after that 2-week period to a group. Subjects are assigned to a treatment group (either one of the active groups or placebo) and continuously receive study drug for a 4 or 12-week period. Subjects continue, as they did during the screening period, to record abdominal pain/discomfort and other lower GI symptoms throughout the 4 or 12-week period. Following completion of the treatment period subjects continue to be record symptoms during a 2-week minimum follow-up period with on-going monitoring.

Endpoints include measurement of adequate relief of abdominal pain/discomfort, a comparison of the proportion of pain/discomfort-free days during the treatment period, change in stool consistency, change in stool frequency and change in gastrointestinal transit.

Exemplification

The present invention will now be illustrated by the following Examples, which are not intended to be limiting in any way.

Materials and Methods

Differential Scanning Calorimetry (DSC)

DSC data was collected on a TA instrument Q1000 equipped with a 50 position autosampler. The energy and temperature calibration standard was indium. Samples were heated at a rate of 10° C./min typically between 25 and 300° C. A nitrogen purge at 30ml/min was maintained over the sample. Between 0.5 and 3 mg of sample was used, unless otherwise stated, and all samples were crimped in a hermetically sealed aluminum pan with a pin hole unless otherwise stated.

Thermogravimetric analysis (TGA)

TGA data was collected on a TA Instrument Q500 TGA, calibrated with Nickel/Alumel and running at a scan rate of 10° C./minute. A nitrogen purge at 60ml/min was maintained over the sample. Typically 5-10 mg of sample was loaded onto a pre-tared platinum crucible.

Polarised Light Microscopy (PLM)

Samples were studied on a Leica LM/DM polarised microscope with a digital camera for image capture. Small amounts of sample were placed on a glass slide with individual particles separated as well as possible and viewed with 50-500× magnification and cross-polarized light coupled to a λ false-color filter.

¹HNMR

All spectra were collected on a Bruker 400 MHz equipped with an autosampler. Samples were prepared in d₆-DMSO, unless otherwise stated.

X-Ray Powder Diffraction (XRPD)—Bruker AXS C2 GADDS Diffractometer

X-ray powder diffraction patterns for the samples were acquired on a Bruker AXS C2 GADDS diffractometer using CuKα radiation (40 kV, 40 mA), automated XYZ stage, laser video microscope for auto-sample positioning and a HiStar 2-dimensional area detector. X-ray optics consists of a single Göbel multilayer mirror coupled with a pinhole collimator of 0.3 mm.

Beam divergence, i.e. the effective size of the X-ray beam on the sample, was approximately 4 mm. A θ-θ continuous scan mode was employed with a sample to detector distance of 20 cm which gives an effective 2θ range of 3.2-29.8°. A typical exposure time of a sample was about 120 s.

Samples run under ambient conditions were prepared as flat plate specimens using powder without grinding unless otherwise stated. Approximately 1-2 mg of the sample was lightly pressed on a glass slide to obtain a flat surface. Samples run under non-ambient conditions (VT-XRPD) were mounted on a silicon wafer with heat conducting compound. The sample was then heated to the appropriate temperature at about 20° C./minute and subsequently held isothermally for about 1 minute before data collection was initiated.

Solubility

Each sample was suspended in 0.25 ml of solvent (water) in an amount effective to produce a maximum final concentration of greater than or equal to about 10 mg/ml of the parent free form of the compound (i.e., adding excess sample to account for the weight of the salt). The suspension was then equilibrated at 25° C. for 24 hours followed by a pH check and filtration through a glass fibre C 96 well plate. The filtrate was then diluted down 101 times. HPLC using a generic 5 minute method was used to quantify the amount of sample in solution with reference to a standard dissolved in DMSO at 0.1 mg/ml. Various volumes of the standard, diluted and undiluted tests were injected. The solubility was then calculated by integration of the peak area found at the same retention time as the peak maximum in the standard injection. If sufficient solid remained in the filter plate the XRPD was normally checked for phase changes, hydrate formation, amorphization, crystallization and other changes.

### EXAMPLE 1

### Characterization of the Hydrochloride

Initial optical inspection showed a white powder which demonstrated birefringence/crystallinity and appeared to consist of small irregular agglomerated particles.

Form I was characterized by pKa, LogP, LogPᵢₒₙ and LogD determination and prediction, XRPD before and after storage at 40° C./75% RH for 4 weeks, PLM, DSC and TGA, ¹HNMR, solubility in water with pH measurement, HPLC purity before and after storage at 40° C./75% RH for 4 weeks, water content, GVS with XRPD and purity analysis after measurement, and VT-XRPD.

The slope of the Yasuda-Shedlovsky extrapolation for potentiometric determination of pKa in MeOH/water indicated that the pKa at 8.64 was basic. A second basic centre with a pKa of 0.81 was measured by UV spectroscopy (D-PAS attachment). As expected from the predicted values, the compound is suitable for a mono salt screen and should form strong stable salts.

LogP, LogPᵢₒₙ and LogD values were measured by using different ratios of 0.1 5M KCl(Aq) to 1-octanol, with refinement of a multiset of the data. The measured LogP, 3.96, is in good agreement with the predicted values. LogPᵢₒₙ was 1.41 and LogD at pH 7.4 was 2.72.

XRPD analysis on both the C2 and D5000 machines showed that the material was highly crystalline. Preparations of substantially pure form I showed an absence of a peak at 4.0 2θ. After storage at 40° C./75% RH for four weeks the XRPD pattern was unchanged. PLM images showed that the material consists of small irregular crystalline particles of up to ˜20 m size.

The initial purity of the material was 100%. However after 4 weeks at 40° C./75% RH under ambient light conditions the material surface had gone yellow and gave a HPLC purity of 99.0%.

The DSC thermogram of form I shows an initial endotherm between 20° C. and about 80° C. due to water loss, followed by a melting endotherm at 269.1° C. (ΔH=113 J/g). The TGA thermogram shows a weight loss of 4.8% between 20° C. and about 70° C. which is equivalent to 1.0 moles of water. Coulometric water determination gave a value of 4.8% which confirmed the DSC and TGA interpretations.

VT-XRPD analysis indicated that the compound might sublime before the melt. The DSC in a hermetic pan with a pin hole shows a shift in the baseline through the melt endotherm, indicative of either degradation or compound loss. Measurement in a hermetic pan without a pin hole does not show a shift in the baseline. The TGA thermogram also shows weight loss from approximately 230° C. The melting point shown in the hermetic pan was 273.6° C. (AH=98 J/g).

¹HNMR analysis in DMSO and CD₃OD was consistent with the given structure, with all 16 protons being identified. The proton in the thiophene ring was set to one in the ¹HNMR integrations and was subsequently used in the salt selection study for integration purposes.

A single cycle of GVS showed a total weight increase of 5.0% between 0 and 90% RH and in particular a weight increase of about 4.8% between 20% and 30% RH which is equivalent to 1.0 moles of water. This weight increase was not associated with any significant hysteresis between the adsorption and desorption cycles. XRPD reanalysis after GVS did not show any change.

Aqueous solubility determination gave a value of 2.1 mg/ml (as the hydrochloride, equivalent to 1.7 mg/ml as the free base) and a pH of 6.33.

### EXAMPLE 2

### Salt Studies

The free base of 4-(2-fluorophenyl)-6-methyl-2-(piperazin-1-yl)thieno[2,3-d] pyrimidine hydrochloride was prepared using known methods. For example, the free base can be made by adding NaOH to a solution of the hydrochloride.

A set of 12 acidic counter-ions were chosen based on pharmaceutical acceptability (class one and two only), pKa of acid (generally at least 2 units lower than the base), occurrence in marketed drugs and likelihood that the salt would increase solubility. HCl was not used in this study since its properties have already been investigated.

The salt formers selected for the screen (class 1 unless stated) were sulphuric acid, methane sulphonic acid (class 2), maleic acid, phosphoric acid, L-tartaric acid, fumaric acid, citric acid, L-malic acid, L-lactic acid, adipic acid, acetic acid, and propionic acid (class 2) 780 mg of free base was dissolved in 6.5 mls of THF with warming to prevent crystallization. 12×0.5 ml of this solution (60 mg of free base) was added to a rack of HPLC vials. 1.11 equivalents of a 1 M solution of each acid in THF was added to the vials. Fumaric acid was added as a warm 0.5M solution due to insufficient solubility in THF. The times taken for a precipitate to form varied from immediate to 15 minutes. An additional 0.4 mls THF was added to the 10 vials with a precipitate to aid mobility of the material since a viscous suspension had formed. The samples were then matured between RT and 50° C. on a 4 hr cycle for 24 Hrs on a shaker. The L-lactate and propionate experiments did not produce a precipitate after this time and were subjected to additional treatment as discussed below.

The L-lactate and propionate were placed at the back of the fumehood with a loosened lid to allow evaporation in an attempt to form a solid. After evaporation the L-lactate and propionate produced mainly an oil/gum with a few tiny non-isolatable crystals in the vial. A few small scale experiments were then attempted on the L-lactate and propionate gums using anti solvents (toluene, hexane, ACN, MEK and MTBE) in an attempt to solidify the material. MTBE with the propionic acid salt on a small scale and scratching produced an instant precipitate. Also, after a longer period a solid was produced from the L-lactate using an MEK-Hexane mixture. MTBE was used on both gums to give precipitation as follows: To the L-lactate vial: (1) 1 ml MTBE was added +200 ul THF+heat to give a solution, (2) the vessel was scratched to give an immediate precipitate; and (3) 1 ml more MTBE was added. To the propionate vial: (1) 1 ml MTBE was added and (2) vessel was scratched to give an immediate precipitate (crystalline by microscopy).

Extra THF was added to 5 viscous suspensions to aid extraction of the material from the vial. The 12 solids produced were filtered off through a 5 micron filter cartridge and dried at 30° C. for 2 hours under vacuum.

Characterization of the Salts

Each of (a) XRPD before and after storage for 4 and 11 days at 40° C./75% RH, (b) PLM (Polarised Light Microscopy), (c) ¹HNMR in DMSO to check for salt formation, stoichiometry and residual THF, and (d) solubility in water at up to 10 mg/ml with pH measurement after equilibration and XRPD of any remaining solid were carried out on the 12 solids from the salt formation experiments.

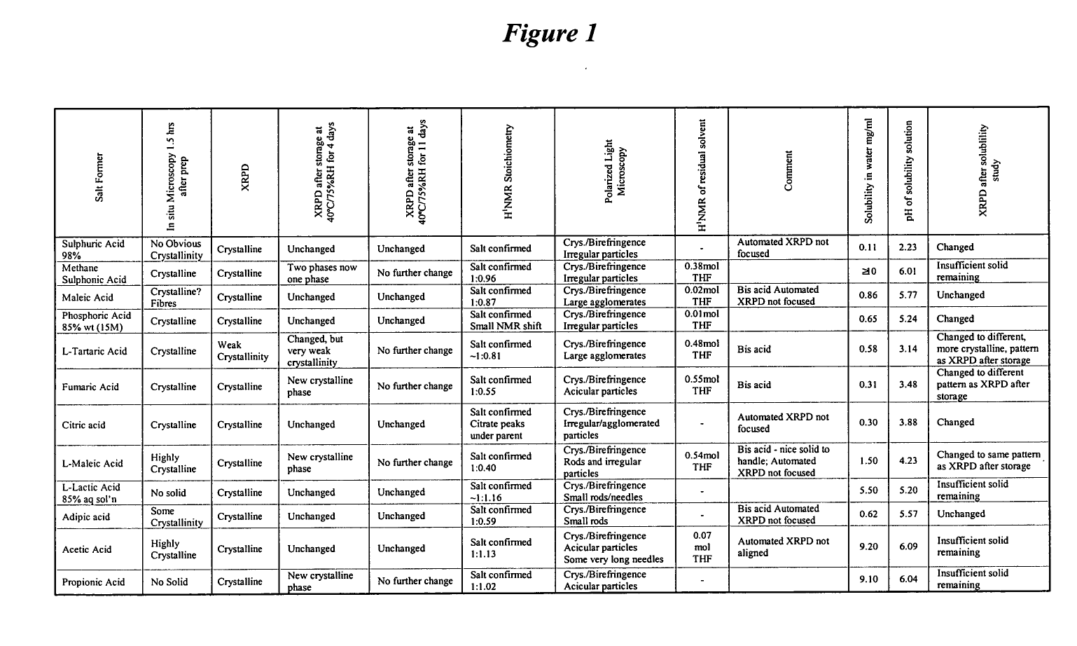

A summary of the characterization data above can be found in FIG. 1 which includes the stoichiometry of the salt from ¹HNMR and THF content. Polarised light microscopy (measurements made at ×20 and ×50 objective magnification) showed that all the materials showed birefringence/ crystallinity with variable crystal habit/size and degrees of agglomeration. HNMR characterization showed that the fumarate, malate and adipate bis acids formed a stoichiometry of 1:0.5.

¹HNMR characterization also showed that four of the crystalline materials retained significant amounts of THF even after drying: mesylate, tartrate, fumarate and malate.

All the materials gave good or very high crystallinity apart from the tartrate salt which was poorly crystalline. Storage of the materials at 40° C./75% RH for 4 days gave a change in the XRPD pattern for the mesylate, tartrate, fumarate, malate, and propionate salts, indicating either hydrate formation or polymorphism of those materials.

Photographs of the solids after 4 and 11 days at 40° C./75% RH showed that some of them were beginning to yellow, similar to the hydrochloride. However, some of the solids had not changed color.

Of those solubility experiments where sufficient material remained, the following salts gave a change in the XRPD pattern compared to the initial form of the sample; sulphate, phosphate, tartrate, fumarate, citrate and malate. This may indicate the formation of a hydrate. The mesylate, L-lactate, acetate and propionate salts had higher solubility in water than the hydrochloride salt.

### EXAMPLE 3

### Treatment of Overactive Bladder Using 4-(2-fluorophenyl)-6-methyl-2-(piperazin-1-yl)thieno[2,3-d] pyrimidine (MCI-225)

The effect of the administration of MCI-225 was assessed using the Dilute Acetic Acid Model. Specifically, the ability of MCI-225 to reverse the irritation-induced reduction in bladder capacity caused by continuous intravesical infusion of dilute acetic acid was assessed.

Dilute Acetic Acid Model-Rats

Female rats (250-275 g BW, n=8) were anesthetized with urethane (1.2 g/kg) and a saline-filled catheter (PE-50) was inserted into the proximal duodenum for intraduodenal drug administration. A flared-tipped PE-50 catheter was inserted into the bladder dome, via a midline lower abdominal incision, for bladder filling and pressure recording and secured by ligation. The abdominal cavity was moistened with saline and closed by covering with a thin plastic sheet in order to maintain access to the bladder for emptying purposes. Fine silver or stainless steel wire electrodes were inserted into the external urethral sphincter (EUS) percutaneously for electromyography (EMG). Animals were positioned on a heating pad which maintained body temperature at 37° C.

Saline and all subsequent infusates were continuously infused at a rate of about 0.055 ml/min via the bladder filling catheter for about 60 minutes to obtain a baseline of lower urinary tract activity (continuous cystometry; CMG). At the end of the control saline cystometry period, the infusion pump was stopped, the bladder was emptied by fluid withdrawal via the infusion catheter and a single filling cystometrogram was performed using saline at the same flow rate as the continuous infusion, in order to measure bladder capacity. Bladder capacity (ml) was calculated as the flow rate of the bladder filling solution (ml/min) multiplied by the elapsed time between commencement of bladder filling and occurrence of bladder contraction (min).

Following the control period, a 0.25% acetic acid solution in saline (AA) was infused into the bladder to induce bladder irritation. Following 30 minutes of AA infusion, 3 vehicle injections (10% TWEEN 80 in saline, 1 ml/kg dose) were administered intraduodenally at 20 minute intervals to determine vehicle effects on the intercontraction interval and to achieve a stable level of irritation with the dilute acetic acid solution. Following injection of the third vehicle control, bladder capacity was again measured, as described above but using AA to fill the bladder. Increasing doses of MCI-225 (3, 10 or 30 mg/kg, as a 1 ml/kg dose) were then administered intraduodenally at 60 minute intervals in order to construct a cumulative dose-response relationship. Bladder capacity was measured as described above using AA to fill the bladder, at 20 and 50 minutes following each subsequent drug treatment.

Data Analysis

Bladder capacity was determined for each treatment regimen as described above (flow rate of the bladder filling solution (ml/min) multiplied by the elapsed time between commencement of bladder filling and occurrence of bladder contraction (min)) and converted to % Bladder Capacity normalized to the last vehicle measurement of the AA/Veh 3 treatment group. Data were then analyzed by non-parametric ANOVA for repeated measures (Friedman Test) with Dunn's Multiple Comparison test. All comparisons were made from the last vehicle measurement (AA/Veh 3). The 30 and 60 minute post-drug measures were very similar, so the average of these two measures was used as the effect for each dose. P<0.050 was considered significant.

Results

Intraduodenal MCI-225 resulted in a dose-dependent increase in bladder capacity in the dilute acetic acid model, as measured by filling cystometry in rats (n=8) during continuous irritation. This effect was statistically significant at the dose range of 3-30 mg/kg (p=0.0005 by Friedman test), the 10 mg/kg and 30 mg/kg responses were significantly higher than AANeh 3 (p<0.05 and p<0.001 by Dunn's multiple comparison test, respectively).

Conclusion

The ability of MCI-225 to reverse the irritation-induced reduction in bladder capacity suggests both a direct effect of this compound on bladder C-fiber activity via 5HT₃ receptor antagonism and an enhancement of sympathetic inhibition of bladder activity via noradrenaline reuptake inhibition. The effectiveness of MCI-225 in this model is predictive of efficacy in the treatment of lower urinary tract disorders in humans.

Dilute Acetic Acid Model-Cats

The ability of MCI-225 to reverse the reduction in bladder capacity seen following continuous infusion of dilute acetic acid in a cat model, a commonly used model of overactive bladder (Thor and Katofiasc, 1995, J. Pharmacol. Exptl. Ther. 274: 1014-24).

Materials and Methods

Six alpha-chloralose anesthetized (50-100 mg/kg) normal female cats (2.5-3.5 kg; Harlan) were utilized in this study.

Drugs and Preparation

MCI-225 was dissolved in 5% methylcellulose in water at 3.0, 10.0 or 30 mg/ml Animals were dosed by volume of injection=body weight in kg.

Acute Anesthetized In Vivo Model

Female cats (2.5-3.5 kg; Harlan) had their food removed the night before the study. The following morning, the cats were anesthetized with isoflurane and prepped for surgery using aseptic technique. Polyethylene catheters were surgically placed to permit the measurement of bladder pressure, urethral pressure, arterial pressure, respiratory rate as well as for the delivery of drugs. Fine wire electrodes were implanted alongside the external urethral anal sphincter. Following surgery, the cats were slowly switched from the gas anesthetic isoflurane (2-3.5%) to alpha-chloralose (50-100 mg/kg). During control cystometry, saline was slowly infused into the bladder (0.5-1.0 ml/min) for 1 hour. The control cystometry was followed by 0.5% acetic acid in saline for the duration of the experiment. After assessing the cystometric variables under these baseline conditions, the effects of MCI-225 on bladder capacity were determined via a 3 point dose response protocol.

Data Analysis

Data was analyzed using a non-parametric One-Way ANOVA (Friedman Test) with the post-hoc Dunn's multiple comparison t test. P<0.05 was considered significant.

Results and Conclusions

MCI-225 caused a significant dose-dependent increase in bladder capacity following acetic acid irritation (P<0.0103), with individual dose significance attained at the 30 mg/kg dose (P<0.05). These data support the initial positive findings in the rat, demonstrating that MCI-225 is effective in increasing bladder capacity in commonly utilized models of OAB in two species. These results are also predictive of the efficacy of MCI-225 in the treatment of BPH, for example, the irritative symptoms of BPH.

### EXAMPLE 4

### Evaluation of MCI-225 in a Model of Visceromotor Response to Colorectal Distension: Treatment of IBS using MCI-225

The ability of MCI-225 to reverse acetic acid-induced colonic hypersensitivity in a rodent model of irritable bowel syndrome was assessed. Specifically, the experiments described herein investigated the effect of MCI-225 on visceromotor responses in a rat model of acetic acid-induced colonic hypersensitivity in the distal colon of non-stressed rats.

Adult male Fisher rats were housed (2 per cage) in the animal facility at standard conditions. Following one week of acclimatization to the animal facility, the rats were brought to the laboratory and handled daily for another week to get used to the environment and the research associate performing the experiments.

Visceromotor Responses to Colorectal Distension (CRD)

The visceromotor behavioral response to colorectal distension was measured by counting the number of abdominal contractions recorded by a strain gauge sutured onto the abdominal musculature as described in Gunter et al., Physiol. Behav., 69(3): 379-82 (2000) in awake unrestrained animals. A 5 cm latex balloon catheter inserted via the anal canal into the colon was used for colorectal distensions. Constant pressure tonic distensions were performed in a graded manner (15, 30 or 60 mmHg) and were maintained for a period of 10 min and the numbers of abdominal muscle contractions were recorded to measure the level of colonic sensation. A 10 min recovery was allowed between distensions.

Acetic Acid-Induced Colonic Hypersensitivity

Acetic acid-induced colonic hypersensitivity in rats has been described by Langlois et al., Eur. J. Pharmacol., 318: 141-144 (1996) and Plourde et al., Am. J. Physiol. 273: G191-G196 (1997). In the present study, a low concentration of acetic acid (1.5 ml, 0.6%) was administered intracolonically to sensitize the colon without causing histological damage to the colonic mucosa as described in previous studies (Gunter et al., supra).

Testing

MCI-225 (30 mg/kg; n=6) or vehicle alone (n=4) were administered to the rats intraperitoneally (i.p.) 30 min prior to initiation of the protocol for colorectal distension. Injection volume was 0.2 mL using 100% propylene glycol as the vehicle. Three consecutive colorectal distensions at 15, 30 or 60 mmHg applied at 10-min intervals were recorded. Visceromotor responses were evaluated as the number of abdominal muscle contractions recorded during the 10-min periods of colorectal distension. Non-sensitized and sensitized uninjected control animals served to demonstrate the lower and upper levels of response, respectively (n=2/group).

Results

Acetic acid reliably sensitized rat visceromotor responses to CRD. Vehicle alone had no effect on the response to CRD in acetic acid sensitized animals. MCI-225 at 30 mg/kg eliminated the visceromotor response to CRD in 50% of the animals.

Conclusion

MCI-225 was shown to be effective in a rat model which can be predictive of drug effectiveness in treating IBS in humans. Specifically, MCI-225 significantly reduced colorectal sensitization-induced increases in visceromotor responses to colorectal distension in 50% of the animals tested.

### EXAMPLE 5

### Comparison of MCI-225, Ondansetron and Nisoxetine in a Model of Visceromotor Response to Colorectal Distension

Additional studies to compare the effects of MCI-225, ondansetron and nisoxetine in the animal model of visceromotor behavioral response to colorectal distension described in Example 4 were conducted.

Adult male rats were used in the study. Similar to Example 4, acute colonic hypersensitivity was induced by intracolonic administration of acetic acid and evaluated as an increased number of reflex abdominal muscle contractions induced by colorectal distension. Specifically, rats were anesthetized with Isoflurane (2%) and were instrumented with a strain gauge force transducer for recording of abdominal muscle contractions. A latex balloon and catheter were inserted 11 cm into the colon. The animals were allowed a 30-min period to completely recover from the anesthesia and were then subjected to intracolonic infusion of acetic acid (1.5 mL, 0.6%). An additional 30-min period was allowed for sensitization of the colon. At the end of this period, animals received a single dose of either MCI-225 or one of the reference drugs or vehicle via intraperitoneal injection. The protocol for colorectal distension was initiated 30-min post drug administration. After a basal reading of the number of abdominal contractions with the balloon inserted but not distended, three consecutive 10-min lasting colorectal distensions at 15, 30, and 60 mmHg were applied at 10-min intervals. Colorectal sensitivity was evaluated by counting the number of reflex abdominal contractions (i.e. the visceromotor response) observed within each distension period.

Animals were randomly assigned to three test groups and dose-dependent controlled experiments were performed as illustrated in Table 1. A control group of animals undergoing the same procedures was treated with vehicle only. Data were summarized for each dose.

TABLE 1

| Treatment Group | Dose (i.p.) | | Number of Subjects |
|---|---|---|---|
| MCI-225 | 3 | mg/kg | 6 |
| MCI-225 | 10 | mg/kg | 6 |
| MCI-225 | 30 | mg/kg | 6 |
| Ondansetron | 1 | mg/kg | 5 |
| Ondansetron | 5 | mg/kg | 5 |
| Ondansetron | 10 | mg/kg | 5 |
| Nisoxetine | 3 | mg/kg | 6 |
| Nisoxetine | 10 | mg/kg | 6 |
| Nisoxetine | 30 | mg/kg | 6 |
| Vehicle (100% Propylene Glycol) | 200 | μL | 11 |

Test and Control Articles

Control drugs for this study were ondansetron and nisoxetine. Ondansetron was supplied from APIN Chemicals LTD. Nisoxetine was supplied by Tocris. MCI-225 was provided by Mitsubishi Pharma Corp. All drugs were dissolved in a vehicle of 100% Propylene Glycol (1,2-Propanediol) by sonicating for a period of 10 minutes. Propylene Glycol was obtained from Sigma Chemical Co.

Adult male Fisher rats were used in this study. The animals were housed two per cage at standard conditions (12 hr light/dark cycle, free access to food and water). Following one week of acclimatization to the animal facility, the animals were brought to the laboratory for a second week and handled by the research associate that preformed the experiments. This allowed the animals to become acclimatized to both the experimental environment as well as the research associate who preformed the experiments. All testing procedures used in the study were preapproved.

Acetic Acid-Induced Colonic Hypersensitivity.

Acetic acid-induced colonic hypersensitivity in rats has been described by Langlois et al. and Plourde et al., referenced above. In this study low-concentration acetic acid (1.5 mL, 0.6%) was administered intracolonically to sensitize the colon without causing histological damage to the colonic mucosa as described in Example 4.

Visceromotor Responses to Colorectal Distension.

The visceromotor behavioral response to colorectal distension was measured by counting the number of abdominal contractions recorded by a strain gauge sutured onto the abdominal musculature as previously described Gunter et al., referenced above. Colorectal distensions were carried out utilizing a 5 cm latex balloon catheter inserted into the colon via the anal canal. Constant pressure tonic distensions were performed in a graded manner, i.e., the pressure was increased to the desired level of 15, 30, or 60 mmHg and then maintained for a period of 10 minutes during which the number of abdominal contractions were recorded to measure the level of colonic sensation. Ten minute recovery periods were allowed following each distension.

Results and Discussion

In nave rats, colorectal distensions at graded intraluminal pressure (0, 15, 30 and 60 mmHg) applied for 10 min. with 10 min. intervals between distensions evoked pressure-dependent visceromotor responses. Acetic acid-induced colonic hypersensitivity was characterized by a pressure-dependent linear increase in the number of abdominal contractions compared to non-sensitized controls. In the present study, rats were treated with the test or reference compounds following colorectal sensitization, thus the obtained drug effects reflect interactions with mechanisms altering the hyper-responsiveness to colonic stimulation without having a preventing effect on the development of colorectal hypersensitivity.

Effect of the Reference Compounds

Ondansetron, a selective 5-HT₃ receptor antagonist, administered at doses of 1, 5, or 10 mg/kg, induced a dose-dependent decrease in the number of abdominal contractions. Ondansetron shows a significant dose-dependent inhibition of the visceromotor response at all distension pressures compared to the effect of the vehicle. However, even the highest dose of 10 mg/kg ondansetron did not abolish the responses to moderate (30 mmHg) and high (60 mmHg) intraluminal pressure, but rather reduced these responses to levels characteristic for nave non-sensitized rats. No significant changes in the behavioral activity of the rats were observed following ondansetron treatment.

Nisoxetine, which acts as an inhibitor of noradrenaline re-uptake, had no significant effect on the visceromotor response to colorectal distension when administered at doses of 3, 10 or 30 mg/kg. However, the high dose of 30 mg/kg nisoxetine was associated with increased exploratory behavior in the home cage during the experiments.

Effect of MCI-225

Compared to the vehicle, MCI-225 administered at a dose of 10 mg/kg caused a significant decrease in the number of abdominal contractions recorded in response to colorectal distension at 15, 30 and 60 mmHg. However, the effects of MCI-225 did not show a normal dose-dependent relationship since the high dose of 30 mg/kg MCI-225 appeared to be less effective. In comparison with the reference compounds, the maximal inhibition of visceromotor responses induced by 10 mg/kg MCI-225 was similar to the inhibition caused by 5 mg/kg ondansetron.

Statistical Analysis

Statistical significance of the treatment groups was assessed using one-way ANOVA followed by Tukey post-test. Differences between responses observed in vehicle treated and drug treated rats were considered significant at p<0.05. (*) p<0.05, (**) p<0.01, (***) p<0.001

Conclusion

MCI-225, was shown to be effective in a rat model which can be predictive of drug effectiveness in treating IBS in humans. Specifically, MCI-225 significantly reduced the number of abdominal contractions recorded in response to colorectal distension at various pressures. Thus, MCI-225 can be used as a suitable therapy for IBS.

### EXAMPLE 6

### Effect of MCI-225 in a Model of Increased Colonic Transit

The model used in this example provided a method of determining the ability of MCI-225 to normalize accelerated colonic transit induced by water avoidance stress (WAS). Ondansetron (5-HT₃ receptor antagonist), nisoxetine (NARI) and a combination of ondansetron and nisoxetine were used as comparison compounds. The model provides a method of evaluating the effectiveness of a compound in a specific patient group of IBS sufferers where stress induced colonic motility is considered a significant contributing factor.

Preliminary testing in the water avoidance stress model confirmed that there exists an association between stress and altered colonic motility. Fecal pellet output was measured by counting the total number of fecal pellets produced during 1 hour of WAS. Using the WAS model, the effect of MCI-225 was compared to the effects of ondansetron (5-HT₃ antagonist) or nisoxetine (noradrenaline reuptake inhibitor -NARI) to affect fecal pellet output. The results showed that MCI-225 inhibited stress-induced accelerated colonic transit and can therefore be effective in the treatment of IBS, particularly IBS where stress induced colonic motility is considered a significant contributing factor.

Adult male F-344 rats, supplied by Charles River Laboratories and weighing 270-350 g, were used to complete this study. The rats were housed 2 per cage under standard conditions. Following one to two weeks of acclimatization to the animal facility, the rats were brought to the laboratory and handled daily for another week to acclimatize them to laboratory conditions and to the research associate who performed the studies. All procedures used in this study were approved in accordance with facility standards.

Acclimatization Prior to Experiments

All rats underwent sham stress (I-hour in stress chamber without water) for 2-4 consecutive days before undergoing WAS (sham was performed until rats produced 0-1 pellet per hour for 2 consecutive days). At the end of the 1-hour stress period, the fecal pellets were counted and recorded.

Procedure

WAS causes an acceleration of colonic transit, which can be quantified by counting the number of fecal pellets, produced during the stress procedure. Rats were placed for 1-hour into a stress chamber onto a raised platform 7.5 cm×7.5 cm×9 cm (L×W×H) in the center of a stress chamber filled with room temperature water 8 cm in depth. The stress chamber was constructed from a rectangular plastic tub (40.2×60.2×31.2 cm). A summary of the treatment and control groups is set forth in Table 2.

TABLE 2

| Treatment Group | Dose (i.p.) | | Number of subjects |
|---|---|---|---|
| MCI-225 | 3 | mg/kg | 8 |
| MCI-225 | 10 | mg/kg | 8 |
| MCI-225 | 30 | mg/kg | 8 |
| Ondansetron | 1 | mg/kg | 8 |
| Ondansetron | 5 | mg/kg | 8 |
| Ondansetron | 10 | mg/kg | 8 |
| Nisoxetine | 3 | mg/kg | 8 |
| Nisoxetine | 10 | mg/kg | 8 |
| Nisoxetine | 30 | mg/kg | 8 |
| Control Group Home Cage | n/a | | 8 |
| Control Group Sham Stress | n/a | | 8 |
| Control Group WAS | n/a | | 8 |
| Control Group Vehicle (100% | 200 | μL | 8 |
| Propylene Glycol) | | | |

Test and Control Articles

Control drugs for this study were ondansetron and nisoxetine. Ondansetron was supplied from APIN Chemicals LTD. Nisoxetine was supplied by Tocris. MCI-225 was provided by Mitsubishi Pharma Corp. All drugs were dissolved in a vehicle of 100% propylene glycol (1,2-propanediol) by sonicating for a period of 10 minutes. Propylene glycol was obtained from Sigma Chemical Co. MCI-225 and nisoxetine were tested at doses of 3, 10 and 30 mg/kg and ondansetron was tested at doses of 1, 5 and 10 mg/kg. All drugs and the vehicle were administered as an i.p. injection in a volume of 0.2 mL.

Results and Discussion

There was no significant difference in the number of fecal pellets produced in 1 hour between the animals in their home cage or the sham stress control group. As expected, upon exposure to a WAS (WAS basal) for 1 hour, there was a highly significant (p<0.001) increase in fecal pellet output compared to fecal pellet output from rats in their home cage or the sham stress control group. After acclimation to the stress chamber for 2-4 days the fecal pellet output of the WAS vehicle treatment group was not statistically different from the fecal pellet output of the non-treated WAS group.

Treatment Groups—MCI-225

In rats pretreated with MCI-225 (dosed at 3, 10 or 30 mg/kg i.p.) and then placed on the WAS, the number of fecal pellets produced during 1 hour was significantly less than the number produced during WAS in the vehicle treated group. MCI-225 caused a significant dose-dependent inhibition of WAS-induced fecal pellet output at all doses.

Nisoxetine

The number of fecal pellets produced during 1 hour of WAS was reduced by all doses (3, 10 and 30 mg/kg i.p.) of Nisoxetine. However, when compared to the vehicle treated group there was significantly fewer fecal pellet produced during WAS at doses of 10 and 30 mg/kg of Nisoxetine.

Ondansetron

Ondansetron caused a dose-dependent inhibition of the stress-induced fecal pellet output. The number of fecal pellets produced during 1 hour of WAS in all ondansetron treatment groups (1, 5 and 10 mg/kg i.p.) was significantly less than the number produced during WAS in the vehicle treatment group.

Combination of Nisoxetine and Ondansetron

For the combination treatment group, the doses of nisoxetine and ondansetron that displayed the most efficacy when dosed alone were used. When nisoxetine (30 mg/kg) was dosed in combination with ondansetron (10 mg/kg), the number of fecal pellets produced during 1 hour of WAS was significantly less than the number produced during WAS in the vehicle control group (p<0.01).

Statistical Analysis

Statistical significance was assessed using one-way ANOVA followed by Tukey post-test. Statistical differences were compared between the WAS groups and the sham stress group and was considered significant if p<0.05. (*) p<0.05, (**) p<0.01, (***) p<0.001

Conclusion

These experiments demonstrated that stress, in this case a water avoidance stressor, caused a significant increase in colonic transit as demonstrated by an increase in fecal pellet output. The overall conclusion is that MCI-225 significantly inhibited the stress-induced increase in fecal pellet production to an extent that resembled that observed with either nisoxetine or ondansetron. Thus, MCI-225 can be used as a suitable therapy for the treatment of non-constipated IBS.

### EXAMPLE 7

### Effect of MCI-225 on Small Intestinal Transit

The effect of MCI-225 on the inhibition of small intestinal transit was evaluated and compared to results obtained using ondansetron, nisoxetine and a combination of ondansetron and nisoxetine using the Small Intestinal Transit Rodent Model described below.

Specifically, the effects of MCI-225, the reference compounds (ondansetron and nisoxetine) and the vehicle on small intestinal transit were investigated in rats under control conditions. Following an overnight fast, rats were brought to the laboratory in their home cages and received an i.p. injection of one of the following: MCI-225, 100% propylene glycol (vehicle), ondansetron, nisoxetine and a combination of ondansetron and nisoxetine. Control rats received no treatment. The treated rats were placed back in the home cages and after 30 min, were fed a 2 mL charcoal meal via an oral gavage. Small intestinal transit was measured following 15 min test-period. Each rat was placed briefly in a glass chamber with IsoFlo for anesthesia and sacrificed. The stomach and the small intestine were removed and the total length of the small intestine was measured. Transit was then measured as the distance that the charcoal meal had traveled along the small intestine and expressed as % of the total length. Animals were randomly assigned to experimental groups and experiments were performed as illustrated in Table 3.

TABLE 3

| | | | | | | Standard |
|---|---|---|---|---|---|---|
| | | | Number | | | Error to |
| | Dose | | of | | Standard | the Mean |
| Treatment Group | (i.p.) | | Subject | Mean | Deviation | (SEM) |
| MCI-225 | 3 | mg/kg | 6 | 30.1% | 20.3% | 8.3% |
| MCI-225 | 10 | mg/kg | 5 | 4.2% | 5.8% | 2.6% |
| MCI-225 | 30 | mg/kg | 4 | 8.8% | 7.1% | 3.5% |
| Ondansetron | 1 | mg/kg | 5 | 27.6% | 16.0% | 7.2% |
| Ondansetron | 5 | mg/kg | 5 | 32.1% | 15.6% | 7.0% |
| Ondansetron | 10 | mg/kg | 5 | 18.4% | 11.8% | 5.3% |
| Nisoxetine | 3 | mg/kg | 6 | 40.3% | 12.2% | 5.0% |
| Nisoxetine | 10 | mg/kg | 5 | 38.6% | 26.7% | 11.9% |
| Nisoxetine | 30 | mg/kg | 5 | 4.7% | 1.05% | 4.7% |
| Nisoxetine and | 10 | mg/kg | 5 | 14.2% | 11.8% | 5.3% |
| Ondansetron | 5 | mg/kg | | | | |
| Control Group | 200 | μL | 5 | 56.0% | 8.0% | 3.6% |
| Vehicle (100% | | | | | | |
| Propylene Glycol) | | | | | | |

| | | | | | |
|---|---|---|---|---|---|
| Naive rats (untreated) | n/a | 5 | 74.6% | 12.4% | 5.6% |

Test and Control Articles

Ondansetron was supplied from APIN Chemicals LTD. Nisoxetine was supplied by Tocris. MCI-225 was provided by Mitsubishi Pharma Corp. All drugs were dissolved in a vehicle of 100% propylene glycol (1,2-Propanediol) by sonicating for 10 min. Propylene glycol was obtained from Sigma Chemical Co. Ondansetron, a 5-HT₃-receptor antagonist was dosed i.p. at 1, 5, and 10 mg/kg. Nisoxetine was administered i.p. at 3, 10, and 30 mg/kg. All doses were delivered in a final volume of 200 μL. Animals in the vehicle control group received 200 μL of 100% Propylene glycol and animals in the normal control group were untreated.

Adult male F-344 rats (230-330 g) were used in the study. The rats were housed 2 per cage under standard conditions. The animals were fed a standard rodent diet and food and water were provided “ad libitum”. Rats were allowed to acclimatize to the animal facility for one week prior to the transit experiments. All procedures used in this study were pre-approved.

Small intestinal transit in rats was investigated by the passage of a charcoal meal along the small intestine during a defined time period (15-min). The animals were deprived of food for 12-16 hrs prior to the experiments. Rats were given a charcoal meal (a mixture of charcoal, gum arabic, and distilled water) as a 2 mL oral gavage and were sacrificed after a 15-min test period. The distance traveled by the charcoal meal was quantified as a percent of the small intestinal length, using the following equation:
Transit (%)=cm traveled by meal/cm total small intestinal length×100

Data and Statistical Analysis

Small intestinal transit was evaluated in relative units (%) of the total intestinal length in the following groups receiving different drug treatment: naive (untreated), vehicle (propylene glycol, 200 μL i.p.), MCI-225 (3, 10 and 30 mg/kg, i.p.), nisoxetine (3, 10 and 30 mg/kg, i.p.), ondansetron (1, 5 and 10 mg/kg, i.p.) and a combination of nisoxetine (10 mg/kg, i.p.) and ondansetron (5 mg/kg, i.p.). A total of 61 experiments were performed (4-6 rats per group).

Statistical analysis was performed to determine mean, standard error to the mean and standard deviation for each group (See Table 3). Differences between individual dose groups within treatments and comparisons between drug-treated and vehicle-treated groups were determined using an unpaired t test considering that when % is used as a relative unit, t-statistic is relevant. In all cases p<0.05 was considered statistically significant.

Results and Discussion

In naive untreated rats the charcoal meal reached a distance of 75±12% of the total length during the 15-min test period. In comparison, when rats received an i.p. injection of the vehicle 30-min prior to receiving the charcoal meal, the small intestinal transit measured under the same conditions was reduced to 56±8% of the total length of the small intestine. However, the vehicle-treated animals showed uniform and reproducible values of small intestinal transport, which served as control to evaluate the effect of drug treatment.

Effect of MCI-225

A series of experiments was performed to establish the effect of increasing doses of 3, 10 or 30 mg/kg MCI-225 on small intestinal transport. Compared to the vehicle, MCI-225 induced a dose-dependent inhibition of small intestinal transit with a maximal reduction of the distance traveled by the charcoal meal to 4.2±2.6% of the total length of the small intestine at a dose of 10 mg/kg.

Effects of the Reference Compounds

In separate studies animals were treated with increasing doses of 1, 10 or 30 mg/kg nisoxetine, which blocks noradrenaline re-uptake. When administered at doses of 3 or 10 mg/kg nisoxetine showed a tendency to decrease small intestinal transit, while a dose of 30 mg/kg almost completely inhibited the transit. The effect of ondansetron administered at doses of 1, 5 or 10 mg/kg was also investigated. Ondansetron caused a significant reduction in small intestinal transit, without showing a normal dose-dependent relationship.

The inhibition caused by nisoxetine was considered the result of delayed stomach emptying, since the charcoal meal was completely retained in the stomach in 4 out of 5 animals following a dose of 30 mg/kg nisoxetine. This effect differed from the effects found with ondansetron or MCI-225, where a portion of the charcoal meal was always found to advance from the stomach into the small intestine. When 5 mg/kg ondansetron and 10 mg/kg nisoxetine were injected simultaneously the drugs showed a reduction in the distance traveled by the meal of 14% of the total length of the small intestine (i.e. the maximal effect of the combination was greater (lower % values) compared to the effects of the individual doses of 5 mg/kg ondansetron or 10 mg/kg nisoxetine). These findings establish that the decrease in small intestinal transit induced by MCI-225 can result from combined effects on 5-HT₃ receptors and noradrenaline re-uptake mechanisms.

### EXAMPLE 8

### Treatment of Vomiting and Retching Using MCI-225

The ability of MCI-225 to reduce retching and vomiting in an accepted model of cytotoxin-induced emesis in the ferret was assessed. Specifically, the experiments described herein investigated the effect of MCI-225 on retching and vomiting induced by cisplatin. Ondansetron was used as a positive control in the model, in view of its known antiemetic activity.

Adult male ferrets (Mustela putario furo) weighing 1200-1880 g were purchased from Triple F Farms (Sayre, Pa.) and housed in individual cages at standardized conditions (12:12 h light/dark cycle and 21-23° C.). Prior to the experiments, the ferrets were allowed a 7-10 day acclimatization period to the animal facility. The ferrets were fed a carnivore diet with free access to food and water throughout the course of the study. The use of the ferret model of emesis and the drug treatment were preapproved in accordance with facility standards.

Cisplatin-Induced Emesis

A cisplatin solution was prepared by adding preheated (70° C.) saline to cisplatin powder (Sigma-Aldrich Co.) and stirring or sonicating at 40° C. until dissolved.

Following administration of the cisplatin and either MCI-225, ondansetron or vehicle alone, the occurrence of retching and vomiting was monitored for a period of 6 hours. Retching was defined as the number of forceful rhythmic contractions of the abdomen occurring with the animal in characteristic posture, but not resulting in the expulsion of upper gastrointestinal tract contents (Watson et al., British Journal of Pharmacology, 115(1): 84-94 (1994)). Vomiting was defined as the forceful oral expulsion of upper gastrointestinal contents. The latency of the retching or vomiting response and the number of episodes were recorded for each animal and summarized for each experimental group (Wright et al., Infect. Immun., 68(4): 2386-9 (2000)).

Drug treatment

Following one hour of acclimation to the observation cage, ferrets received an intraperitoneal (i.p.) injection of cisplatin (5 mg/kg in 5 mL) followed in about 2 minutes by i.p. injection of a single dose of MCI-225 or ondansetron (Rudd and Naylor, Eur. J. Pharmacol., 322: 79-82 (1997)). Dose-response effects of MCI-225 dosed at 1, 10 and 30 mg/kg i.p. in a 0.5 mL/kg solution or ondansetron dosed at 5 and 10 mg/kg i.p. in a 0.5 mL/kg solution were studied. Each animal received a single-dose drug treatment. In addition, three animals received an initial dose (30 mg/kg i.p.) and a second MCI-225 injection (30 mg/kg i.p.) 180 minutes following the initial dose. Control animals were treated with cisplatin followed by vehicle alone (propanediol dosed in a 0.5 mL/kg solution). All groups were randomized.

Results—Vehicle Alone

Cisplatin induced an emetic response in 100% of the animals receiving vehicle. The mean response was characterized by a total number of 42.8±8.1 events (both retches and vomits), which occurred during the observation period. The mean latency of the first response was 133±22 min post-cisplatin administration.

Results—Ondansetron

Ondansetron applied at the 5 mg/kg and 10 mg/kg dose-dependently reduced the number of emetic events induced by cisplatin. The effect of ondansetron was accompanied by an increase in the latency of the first emetic response following cisplatin treatment. The results are set forth in Table 4 (*p<0.05).

TABLE 4

| No. of | | Retches | Vomits | Total | Latency |
|---|---|---|---|---|---|
| Animals | Treatment | (360 min) | (360 min) | Events | (min) |
| N = 10 | Vehicle | 42.8 ± 8.1 | 3.3 ± 0.8 | 46.1 ± 7.8 | 133 ± 22 |
| N = 7 | Ondansetron | 11.2 ± 7.0 | 0.3 ± 0.2 | 11.5 ± 7.2 | 288 ± 4 |
| | (5 mg/kg) | | | | |
| N = 7 | Ondansetron | 2.4 ± 1.6 | 0.0 ± 0.0 | 2.4 ± 1.6* | 313 ± 32 |
| | (10 mg/kg) | | | | |

Results -MCI-225

As set forth in Table 5, administration of MCI-225 at concentrations of 1, 10 or 30 mg/1 kg caused dose-dependent reduction in the retches and vomits induced by cisplatin (*p<0.05). The emetic response was eliminated by administration of two doses of 30 mg/kg, applied b.i.d at a 180-min interval. The decrease in the number of emetic events induced by MCI-225 was accompanied by an increase in the latency of the response.

TABLE 5

| No. of | | Retches | Vomits | Total | Latency |
|---|---|---|---|---|---|
| Animals | Treatment | (360 min) | (360 min) | Events | (min) |
| N = 10 | Vehicle | 42.8 ± 8.1 | 3.3 ± 0.8 | 46.1 ± 7.8 | 133 ± 22 |
| N = 10 | MCI-225 (1 mg/kg) | 30.4 ± 9.1 | 2.5 ± 0.7 | 32.9 ± 9.8 | 186 ± 35 |
| N = 10 | MCI-225 (10 mg/kg) | 22.9 ± 10.3 | 2.6 ± 1.0 | 25.5 ± 11.1 | 192 ± 57 |
| N = 11 | MCI-225 (30 mg/kg) | 3.3 ± 2.2 | 0.7 ± 0.5 | 4.0 ± 2.6* | 287 ± 38 |
| N = 3 | MCI-225 (30 mg/kg | 0.0 ± 0.0 | 0.0 ± 0.0 | 0.0 ± 0.0 | 360 ± 0 |
| | b.i.d) | | | | |

Conclusion

The results set forth in Tables 4 and 5 show that MCI-225 is effective at reducing retching and vomiting in an accepted animal model of emesis, using a similar dose range as the positive control (ondansetron). Thus, MCI-225 can be used in the treatment of nausea, vomiting, retching or any combination thereof in a subject.

Equivalents

Those skilled in the art will recognize, or be able to ascertain using no more than routine experimentation, many equivalents to the specific embodiments of the invention described herein. Such equivalents are intended to be encompassed by the following claims.

Incorporation by Reference

The contents of all references, patents, and patent applications cited throughout this application are hereby incorporated by reference.

We claim:

1. A compound selected from the group consisting of highly soluble salts of Formula II: 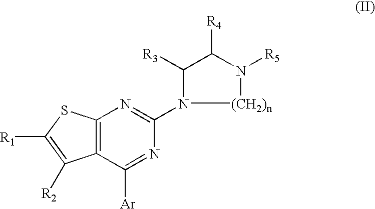
wherein
R₁ and R₂ are each independently hydrogen, halogen or a C₁-C₆ alkyl; or R₁ and R₂ are taken together with the carbons to which they are attached to form a 5 or 6 membered cycloalkylene group;
R₃ and R4 are each independently hydrogen or a C₁-C₆ alkyl;
R₅ is hydrogen, C₁-C₆ alkyl, 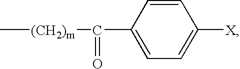 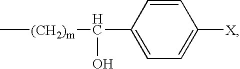
or —C(O)—NH—R₆;
m is an integer from 1 to 3; X is a halogen; and R6 is a C₁-C₆ alkyl;
n is an integer of 2 or 3; and
Ar is a substituted or unsubstituted phenyl, 2-thienyl, or 3-thienyl group,
wherein the solubility of said salt is greater than about 2.0 mg/ml in water as quantified by HPLC.

2. The compound of claim 1, wherein R₁ is -CH₃.

3. The compound of any of the preceding claims, wherein R₂ is hydrogen.

4. The compound of any of the preceding claims, wherein R₃ is hydrogen.

5. The compound of any of the preceding claims, wherein R4 is hydrogen.

6. The compound of any of the preceding claims, wherein R₅ is hydrogen.

7. The compound of any of the preceding claims, wherein n is 2.

8. The compound of any of the preceding claims, wherein Ar is ortho-fluorophenyl.

9. The compound of any of the preceding claims, wherein the compound is a 4-(2-fluorophenyl)-6-methyl-2-(piperazin-1-yl)thieno[2,3-d] pyrimidine salt.

10. The compound of any of the preceding claims, wherein a counterion of the salt has no additional acidic hydrogens.

11. The compound of any of the preceding claims, wherein the salt is at least one salt selected from the group consisting of methane sulphonate, L-lactate, acetate and propionate.

12. The compound of any of the preceding claims, wherein the solubility of the salt is greater than about 2.5 mg/ml in water.

13. The compound of any of the preceding claims, wherein the solubility of the salt is greater than about 5.0 mg/ml in water.

14. The compound of any of the preceding claims, wherein the solubility of the salt is greater than about 7.5 mg/ml in water.

15. The compound of any of the preceding claims, wherein the solubility of the salt is greater than about 10.0 mg/ml in water.

16. A pharmaceutical composition comprising at least one compound of any of the preceding claims and a pharmaceutically acceptable carrier.

17. A method for treating a gastrointestinal tract disorder or a genitourinary disorder in a subject comprising administering to the subject a composition comprising a therapeutically effective amount of a composition of claim 16, such that the gastrointestinal tract disorder or genitourinary disorder is treated.

18. The method of claim 17, wherein the disorder is a functional bowel disorder, irritable bowel syndrome, irritable bowel syndrome with diarrhea, chronic functional vomiting, overactive bladder or a combination thereof.

19. A method for treating an MCI-225 responsive state comprising administering to the subject a therapeutically effective amount of a composition of claim 16, such that the MCI-225 responsive state is treated.
